(19) United States
(12) Patent Application Publication
(10) Pub. No.: US 2011/0002971 A1
(43) Pub. Date: Jan. 6, 2011

(54) CERIA FOR USE AS AN ANTIMICROBIAL BARRIER AND DISINFECTANT IN A WOUND DRESSING

(75) Inventors: Carl R. Hassler, Gig Harbor, WA (US); John L. Burba, Parker, CO (US); Charles Whitehead, Henderson, NV (US); Joe Pascoe, Las Vegas, NV (US)

Correspondence Address: SHERIDAN ROSS PC, 1560 BROADWAY, SUITE 1200, DENVER, CO, 80202, US

(73) Assignee: MOLYCORP MINERALS, LLC, Greenwood Village, CO (US)

(21) Appl. No.: 12/831,054

(22) Filed: Jul. 6, 2010

Related U.S. Application Data
(60) Provisional application No. 61/223,250, filed on Jul. 6, 2009.
(60) Provisional application No. 61/237,148, filed on Aug. 26, 2009.
(60) Provisional application No. 61/255,020, filed on Oct. 26, 2009.

(51) Int. Cl. A61K 9/14; A61K 9/12; A61K 33/24; A01N 25/08; A01P 1/00; A61P 31/00

(52) U.S. Cl. 424/404; 424/489; 424/45; 424/617; 424/402; 424/411; 424/409

(57) ABSTRACT

The present invention relates to protecting a human from an infection using a disinfecting agent as described herein and a method for use thereof, more particularly to a rare earth-containing device for protecting a wound and a method for use thereof.

### CROSS REFERENCE TO RELATED APPLICATIONS

The present application claims the benefits of U.S. Provisional Application Ser. No. 61/223,350, filed Jul. 6, 2009, entitled “Ceria for Use as an Antimicrobial Barrier and Disinfectant in a Wound Dressing”; U.S. Provisional Application Ser. No. 61/237,148, filed Aug. 26, 2009, entitled “Ceria for Use as an Antimicrobial Barrier and Disinfectant in a Wound Dressing”; and U.S. Provisional Application Ser. No. 61/255,025, filed Oct. 26, 2009, entitled “Rare Earth-Containing Nanoparticles and a Method for Making and Using the Same”; which are all incorporated herein by this reference in their entirety.

### FIELD OF INVENTION

The present invention relates to protecting a living organism from an infection using a disinfecting agent as described herein and a method for use thereof, more particularly to a rare earth-containing device for protecting a living organism from infectious matter and a method for use thereof.

### BACKGROUND OF THE INVENTION

Living organisms, such as plants and animals, are susceptible to infection or disease caused by infectious matter. The infectious matter may be a microorganism (such as, bacteria, fungus, or mold), a virus, or a prion.

Infectious matter typically infects a living organism by direct contact with a person or object carrying the infectious matter, by environmental contact (such as, a fluid (air, water or other liquid) or solid carrying the infectious matter), or by self-contamination (such as, in case of animal, physical migration from the animal's skin or gastrointestinal tract).

Disease and/or infection can weaken a living organism. The living organism, in the weakened state, is susceptible to attack by other infectious matter and further disease and infection. In some instances, the disease or infection resulting from the infectious matter can kill the living organism.

An infection or disease caused by a microorganism can be treated with an antibiotic. An antibiotic is commonly a chemical substance, including iodine and silver, having the capacity to inhibit the growth and/or reproduction of and/or kill the microorganism causing the infection or disease.

Elemental iodine, I₂, has antiseptic properties against some infectious matter. Most common forms of antiseptic iodine are: cadexomer iodine (a polysaccharide starch lattice having about 0.9% elemental iodine) and povidone iodine or PVP-1 (an iodophor composed of elemental iodine and a synthetic polymer).

Silver metal and silver compounds have been used as microbials for over a century. Silver compounded with an antibiotic, such as a sulphonamide, is toxic to a broad-spectrum of bacteria and fungi. It is believed that silver can enter a bacterial cell and interfere with one or both of cell multiplication and electron transport.

However, microorganism strains resistant to antibiotics have developed. Treatment regiments available for treating an infection or disease caused by an antibiotic resistant microorganism strain are limited. Furthermore, the overuse of broad-spectrum antibiotics is exacerbating the resistance of microorganisms to antibiotics.

Antibiotics are not effective for treating viral infections and diseases. Viral infections and diseases are typically treated prophylactically by administering a viral-antibody. The viral-antibody provides protection from a specific virus, more specifically the specific virus strain the viral-antibody was developed from. Viruses, however, continually mutate. The viral-antibody typically provides limited, if any, protection against mutant virus forms.

A need exists for more aggressive and effective protection from infectious matter. Also, needed are treatments that provide protection from mutant forms of the infectious matter. Moreover, a less expensive and/or more environmentally friendly treatment for preventing infections and diseases caused by infectious matter are needed.

### SUMMARY OF THE INVENTION

These and other needs are addressed by the various embodiments and configurations of the present invention. This disclosure relates generally to rare-earth antimicrobial compositions, applications for such compositions, and techniques, methodologies, and devices for such applications.

A first embodiment of the present invention comprises contacting one or more rare earth-containing compositions with an infectious biological matter having a first infectious biological matter population. The contacting of the rare earth-containing compositions with the infectious biological matter forms a second infectious biological matter population. The second infectious biological matter population is less the first infectious biological matter population. Furthermore, contacting of the one or more rare earth-containing compositions with the infectious matter includes killing and/or deactivating the infectious biological matter.

A second embodiment of the present invention comprises:

(a) positioning one or more rare earth-containing compositions in a target zone, wherein the target zone has a first population of an infectious biological matter;

(b) contacting the one or more rare earth-containing compositions with the infectious biological matter contained with the target zone; and

(c) killing and/or deactivating the infectious biological matter to form a second population of the infectious biological matter. The second population of the infectious biological matter is less than the first population of the infectious biological matter.

A third embodiment of the present invention comprises: one or more rare earth-containing compositions; and one of: a woven textile; a non-woven textile; an item of apparel; a medical device comprising a textile; a medical device comprising a polymer; a medical device having a polymeric component; a medical implant; a therapeutic formulation; a cleaning composition; a cellulosic-containing material; a polymeric material; a coating material; and an inorganic material.

A fourth embodiment of the present invention comprises:

(a) forming a suspension of a rare earth salt;

(b) charging the suspension to an autoclave;

(c) applying one or both of heat and pressure to the suspension to form an autoclaved suspension;

(d) separating the autoclaved suspension into a liquid phase and a solid phase, and;

(e) calcining one or both of the liquid and solid phases to form rare earth-containing particles. Optionally, the embodiment further comprises sealing the autoclave prior to the applying of one or both of heat and pressure to the suspension. Preferably, the suspension comprises an aqueous suspension. Preferably, the rare earth salt is a substantially insoluble rare earth salt. Optionally, the suspension is substantially quiescent during the applying of the one or both of heat and pressure to the suspension. Preferably, one or both of liquid and solid phases are dried prior to calcining.

Embodiments of the present invention further include at least the following:

Killing and/or deactivating the infectious biological matter is by an interaction of the infectious matter with the one or more rare earth-containing compositions. The interaction is one of a chemical interaction, a physical interaction, or a combination of a chemical and a physical interaction.

The infectious biological matter is one or more of a bacterium, a protozoa, a virus, a fungi, a prion, or a mixture thereof. The infectious biological matter is positioned on or adjacent to an organism.

The target zone is on or about one of an animal or plant. Preferably, the target zone is one of a wound, an infected wound, a surgical area, an area prone to infection, an area to be protected from the infectious biological matter, an area infected and/or diseased with the infectious biological matter, or a combination thereof.

The organism is one of an animal or a plant. The animal is one of a human, a domesticated animal, a wild animal, an animal raised as a source of food or income, a companion animal, or a combination thereof. The plant is one of a cultivated plant, an uncultivated or wild plant, a plant cultivated for nutritional purposes, plants cultivated for non-food purposes, and combinations thereof.

The one or more rare earth-containing compositions comprise particles. In a first particle size embodiment, the particles have a typical average particle size of from about 0.1 nanometers to about 1,000 microns. In a second particle size embodiment, the average particle size is typically from about 0.1 microns to about 10 microns. In a third particle size embodiment, the average particle size is typically from about 1 micron to about 100 microns.

In a fourth particle size embodiment, the rare earth-containing particles, typically, have an average particle size of from about 0.1 microns to about 300 microns. Preferably, about 80% of the particles have an average particle size of from about 0.1 microns to about 2 microns.

In a fifth particle size embodiment, the rare earth-containing particles typically have an average particle size of from about 0.2 microns to about 0.7 microns. Preferably, about 90% of the particles have an average particle size of from about 0.2 microns to about 0.4 microns.

Preferably, the particles have an average particle size of from about 50 nanometers to about 1,000 microns and an average surface area of at least about 1 m²g⁻¹. In a first particle surface area embodiment, the average surface area is more than about 120 m²g⁻¹.

In one embodiment, one of the one or more rare earth-containing compositions comprises cerium. When one of the one or more rare earth-containing compositions comprises cerium, the other of the one or more rare earth-containing compositions comprises one or more rare earth elements selected from the group of rare elements consisting essentially of La, Nd, Pr, and Sm. Preferably, cerium-containing composition includes cerium oxide. More preferably, the cerium-containing composition comprises one or more of cerium (IV) oxide (CeO₂) and cerium (III) oxide (Ce₂O₃).

Optionally, the one or more rare earth-containing compositions contains a water soluble rare earth-containing composition. The water soluble composition preferably has a total dissolved rare earth concentration of about 1 M or more, more preferably of about 1×10⁻¹ M or more, even more preferably of about 5×10⁻² M or more, of at least about 1×10⁻² M, and even more preferably of about 1×10⁻³ M or more.

Optionally, the one or more of the rare earth-containing compositions comprise an insoluble rare earth-containing composition. The water insoluble composition preferably has a total dissolved rare earth concentration of less than about 5×10⁻² M, more preferably of less than about 1×10⁻² M, even more preferably of less than about 1×10⁻³ M, even more preferably of less than about 1×10⁻⁴ M, even more preferably of less than about 1×10⁻⁵ M, even more preferably of less than about 1×10⁻⁶ M, even more preferably of less than about 1×10⁻⁷ M, even more preferably of less than about 1×10⁻⁸ M, even more preferably of less than about 1×10⁻⁹ M, and even more preferably of less than about 1×10⁻¹⁰ M.

The one or more rare earth-containing compositions are contained within a device. The device is one or more of a textile, an item of apparel, a medical device, a therapeutic formulation, a cleaning composition, a cellulosic-containing material, a polymeric material, a coating material, an inorganic material, a woven or non-woven textile, or a combination thereof. The item of apparel is worn by an animal, including a human. The cleaning composition is a fluid or solid having at least one surfactant. The cellulosic-containing material comprises one or more of a paper, a cotton, wood, a wood-containing product, or combination thereof. The polymeric product is one of a homo-polymer, co-polymer, block-polymer, polymeric mixture, polymeric alloy, or a combination thereof comprising one or more of a polyacetal, a polyacrylic, a polyanhydride, a polyamide, a polycarbonate, a polydiene, a polyester, a polyhalo-olefin, a polyimide, a polyimine, a polyketone, a polyolefin, a polyoxide, a polyphylene, a polyphosphazene, a polysilane, a polysiloxane, a polystyrene, a polysulfide, a polysulfoamide, a polysulfonate, a polysulfone, a polysulfoxide, a polythianhydride, a polythioamide, a polythiocarbonate, a polythioester, a polythioketone, a polythioimide, a polythiourea, a polythiourethane, a polyurea, a polyurethane, a polyvinyl, cellulose, chitin, keratin, and a combination or mixture thereof. The medical device is one of a suture, gauze, sponge, swab, dressing, drape, bandage, a stapler, surgical instrument, a light-handle cover, medical tubing, medical mesh, an implant, drain component, wound vac component, or combination thereof. The therapeutic formulation is one an aerosol spray, a powder, cream, ointment, slave, liniment, gel, medical solution, wound irrigation system, or combination thereof.

These and other advantages will be apparent from the disclosure of the invention(s) contained herein.

As used herein, the term “a” or “an” entity refers to one or more of that entity. As such, the terms “a” (or “an”), “one or more” and “at least one” can be used interchangeably herein. It is also to be noted that'the terms “comprising”, “including”, and “having” can be used interchangeably.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The preceding is a simplified summary of the invention to provide an understanding of some aspects of the invention. This summary is neither an extensive nor exhaustive overview of the invention and its various embodiments. It is intended neither to identify key or critical elements of the invention nor to delineate the scope of the invention but to present selected concepts of the invention in a simplified form as an introduction to the more detailed description presented below. As will be appreciated, other embodiments of the invention are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present invention(s). These drawings, together with the description, explain the principles of the invention(s). The drawings simply illustrate preferred and alternative examples of how the invention(s) can be made and used and are not to be construed as limiting the invention(s) to only the illustrated and described examples.

Further features and advantages will become apparent from the following, more detailed, description of the various embodiments of the invention(s), as illustrated by the drawings referenced below.

FIG. 1 depicts a plan view of a particle or particulate having a shape resembling a sphere according to first embodiment of the present invention;

FIG. 2 depicts a cross-sectional view of a core-shell particle or particulate according to a second embodiment of the present invention;

FIG. 3 depicts a plan view of a particle or particulate resembling a fiber according to third embodiment of the present invention;

FIG. 4 depicts a first process for making particles or particulates according to a first method of the present invention;

FIG. 5 depicts a second process for making particles or particulates according to a second method of the present invention;

FIG. 6 depicts a third process for making particles or particulates according to a third method of the present invention;

FIG. 7 depicts a particle or particulate size distribution according to a first particle or particulate size embodiment of the present invention;

FIG. 8 depicts a particle or particulate size distribution according to a second particle or particulate size embodiment of the present invention;

FIG. 9 depicts a particle or particulate size distribution according to a third particle or particulate size embodiment of the present invention;

FIG. 10 depicts a particle or particulate size distribution according to a fourth particle or particulate size embodiment of the present invention;

FIG. 11 depicts a particle or particulate size distribution according to a first control sample of Example IV of the present invention;

FIG. 12 depicts a particle or particulate size distribution according to a first calcinated control sample of Example IV of the present invention;

FIG. 13 depicts a particle or particulate size distribution according to a first control aqueous sample of Example IV of the present invention; and

FIG. 14 depicts a particle or particulate size distribution according to a sonicated control sample of Example IV of the present invention.

### DETAILED DESCRIPTION OF THE INVENTION

Embodiments of the present invention are directed to a disinfecting agent and methods for using the disinfecting agent to reduce infectious matter populations within a target zone. More specifically, the present invention includes the use of the disinfecting agent to reduce infectious matter populations in the target zone on or about a living organism. In particular, the disinfecting agent is contacted with the infectious matter about the target zone. Before discussing the present invention in more detail and to provide content for the discussion of the invention, infectious matter, living organism, target zone, and disinfecting agent will be described in more detail.

### Infectious Matter

As used herein, “infectious matter” refers to any animate (having life) or inanimate (lacking life) biological matter capable of causing disease, infection or both. Examples of infectious matter are, without limitation, bacteria, protozoa, viruses, funguses (including molds and mildews), and prions.

As used herein, “bacteria” (or in singular form “bacterium”) refer to single-celled or non-cellular spherical (typically referred to as cocci) or spiral (typically referred to as priochates) or rod-shaped (typically referred to bacilli) organism lacking chlorophyll and reproducing by fission. Bacteria can be beneficial, benign, or pathogenic to a living organism. Unless indicated otherwise, the term “bacteria” used herein refers to bacteria causing a disease and/or infection. Non-limiting diseases caused by bacteria include cholera, syphilis, antrax, leprosy, bubonic plague, and tuberculosis. Non-limiting examples of infectious bacteria are chlamydia, which include, but are not limited to, Escherichia coli, Methicillin resistant Staphylococcus aureus, Chlamydia trachomatis, Providencia stuartii, Vibrio vulnificus, Pneumobacillus, Nitrate-negative bacillus, Staphylococcus aureus, Candida albicans, Bacillus cloacae, Bacillus allantoides, Morgan's bacillus (Salmonella morgani), Pseudomonas maltophila, Pseudomonas aeruginosa, Neisseria gonorrhoeae, Bacillus subtilis, Bacillus foecalis alkaligenes, Streptococcus hemolyticus B, Citrobacter, and Salmonella paratyphi C. Examples of bacterial capable of causing wound infectious are without limitation: beta haemolytic streptococci (Streptococcus pyogenes), Enterococci (Enterococcus facalis), Strphylococci (Staphylococcus aureus/MSRA and Group D), Prseudomanas aeruginosa, Enterobacter species, Escherichia coli, Kiebsiella, Proteus species, Bacteroides (including fragillis), Clostridium, Coagulaes-negative staphylococci, Enterococci, Proteus mirabilis, Candida Albicanus, and gram-positive aerobes.

As used herein, “fungi” refers either single-celled yeasts or multi-cellular organisms with a nucleus contained within a cell membrane. Fungi are typically larger and more complex than bacteria. While not wanting to be limited by example, fungi can cause skin, nail and hair infections. Examples of infections caused by fungi are without limitation: yeast (Candida) and Aspergillus.

The term “protozoa” refers to single celled organisms. Protozoa have a fragile membrane and lack a cell wall. While not wanting to be limited by example, protozoa are associated with skin ulcers, more specifically infected skin ulcers.

As used herein, “virus” refers to genetic material (that is, material comprising a nucleic acid) enclosed within a protein coat or a membranous envelope. While not wanting to be limited by any theory, viruses do not generally cause wound infections. However, skin lesions can form during the course of a viral disease and can become subsequently infected by bacteria.

As used herein, “prion” refers to is a protein that normally occurs in a harmless form, but when folded into an aberrant shape becomes infectious matter. More specifically, as used herein, “prion” refers to the aberrant-shaped prion capable of causing a disease and/or infection. The same protein forming the prion is harmless, when normally shaped, and is a disease and/or infection causing agent, when aberrantly shaped. Prions can cause a number of degenerative brain diseases, including scrapie (a fatal disease of sheep and goats), mad cow disease, Creutzfeldt-Jacob disease, fatal familial insomnia, kuru, an unusual form of hereditary Gertsmann-Straeussler-Scheinker disease, and possibly in some cases of Alzheimer' disease.

### Living Organism

As used herein, “living organism” refers to a member of biological plant or animal kingdoms, such as members of the plant and animal kingdoms within the biological kingdom systems of Haeckel, Copeland, Wittaker, Woese et al., or Cavalier-Smith. The living organism can be domesticated or wild (in the case of a member of the animal kingdom) or cultivated or uncultivated (in the case of a member of the plant kingdom).

A living organism of the animal kingdom refers to any domesticated or wild animal and includes without limitation any companion animal, any animal raised as a source of food or income, any wild animal being treated for compassionate or environmental purpose, and any member of the mammalian class, including humans. More specifically, companion animals can include, without limitation, cats, dogs, horses, ferrets, guinea pigs, reptiles, and birds. Animals raised as a source of food can include, without limitation, cattle, goats, sheep, llamas, pigs, fish, shellfish, chickens, and ostriches. More specifically, a member of the mammalian class includes any animal that is warm blooded, has lungs, has vertebrate and feeds milk to its babies. While not wanting to be limited by example, mammals include without limitation, humans, dogs, cats, horses, cattle, goats, sheep, llamas, pigs, buffalo, bison, and elk. In one preferred embodiment of the present invention, the living organism is a human.

A living organism of the plant kingdom refers to any cultivated or uncultivated plant and includes without limitation, plants cultivated for nutritional purposes and for non-food purposes, and any uncultivated plants being managed for ecological and/or environmental purposes. Plants cultivated for nutritional purposes are plants grown as a source of food, such as without limitation, maze, corn, berries, wheat, rice, tomatoes, peppers, celery, lettuce, cabbage, potatoes, walnuts, almonds, sugar cane, oats, olives, barely, almonds, peanuts, zucchini, beans, oranges, apples, cherries, figs, pears, peaches, grapefruit, and mangoes. Plants cultivated for non-food purpose are plants grown for one or more of a fiber source (such as, without limitation, cotton, trees, and hemp) a fuel (such as, without limitation, trees, olives, corn, and sugar cane) a medical application, a herbal remedy, a chemical product (such as, corn, wheat, oats, and sugar beet and cane), an aesthetic purpose (such as, landscaping and house plants), and a functional application (such as, erosion or soil control).

### Target Zone

As used herein, “target zone” refers to a location, an area, or a volume where the infectious matter is or could be present. In one embodiment, the target zone can be a location, area, or volume having a population of the infectious matter sufficiently large enough to cause disease or infection. More specifically, the target zone is treated, such as with a disinfecting agent, in response to the presence of the infectious matter within the target zone.

In another embodiment, the target zone is a location of, area on, and/or volume about the living organism and is treated prophylactically to protect the living organism from the infectious matter. That is, while the target zone is substantially free of or has a population of the infectious matter sufficiently too small as to cause disease or infection, the target zone is or has the potential to be exposed to infectious matter. The exposure to the infectious matter is sufficiently large enough to cause disease or infection.

Non-limiting examples of target zones are a wound, a burn and/or scald related infection, dermal, mucosal or dental diseased or infected region, an infected wound, a surgical region, a region being prepared for a surgical procedure, a region prone to infection, a region infected with a infectious matter (such as but not limited to vaginitis or acne), an entryway into a living organism, a substance in contact with or being introduced into a region of a living organism, and a region needing protection from a infectious matter.

The term “wound” refers to damage to a tissue or cellular structure caused by trauma or dissection (such as a surgical procedure). The tissue may comprise an organ, the organ's underlying tissue, or both. The organ can be any organ, including any external (such as, skin) or internal organs (such as, endocrine, neurological, circulatory, intestinal or skeletal systems) of animal, or a shoot or a root system of a plant.

The term “infected wound” refers to, without limitation, wounds known within the medical art as “wound contamination” (bacteria present within the wound without a reaction from the infected living organism), “wound colonization” (bacteria present within the wound which have multiplied and have initiated a reaction from the infected living organism), “critical colonization” (bacterial multiplication causing a delay in wound healing and previously unreported exacerbation of pain), and “wound infection” (deposition and multiplication of bacteria in tissue with a reaction from the infected living organism). Furthermore, the term “infected wound” can refer to a type or class of wounds commonly classified as: “clean” uninfected operative wound lacking a visible acute inflammation (also, commonly referred to a class 1 wound); “clean-contaminated” elective entry wound into respiratory, billiary, gastrointestinal tracts with minimal spillage and no evidence of infection or major break in aseptic technique (also, commonly referred to as a class 1 wound); “contaminated” having one or more of nonpurulent inflammation, gross spillage from gastrointestinal tract, penetrating traumatic wound (<4 hrs), and major break in aseptic technique (also, commonly known as a class III wound); and “dirty infected” having one or more of purulent inflammation, preoperative perforation of viscera, and penetrating traumatic wound (>4 hrs).

As used herein “surgical region” refers to the region of the organism where the surgical dissection is conducted and includes all tissues and organs dissected during the surgical procedure and all regions substantially adjacent to any of the dissected tissues and/or organs.

The term “region prepared for a surgical procedure” refers to the region requiring, according to standard surgical techniques, disinfecting prior and/or during dissection. The region can refer to one or more regions on a living organism. Moreover, the region can be an external region, an internal region, or both.

### The Disinfecting Agent

The disinfecting agent comprises one or more rare earth-containing compositions. As used herein, “rare earth” refers to one or more of yttrium, scandium, lanthanum, cerium, praseodymium, neodymium, samarium, europium, gadolinium, terbium, dysprosium, holmium erbium, thulium, ytterbium, and lutetium. As will be appreciated, lanthanum, cerium, praseodymium, neodymium, samarium, europium, gadolinium, terbium, dysprosium, holmium erbium, thulium, ytterbium, and lutetium are known as lanthanoids.

As used herein, a “composition” refers to one or more chemical units composed of one or more atoms, such as a molecule, polyatomic ion, chemical compound, coordination complex, coordination compound, and the like. As will be appreciated, a composition can be held together by various types of bonds and/or forces, such as covalent bonds, metallic bonds, coordination bonds, ionic bonds, hydrogen bonds, electrostatic forces (e.g., van der Waal's forces and London's forces), and the like.

The rare earth-containing composition(s) can comprise a single rare earth composition or two or more differing rare earth-containing compositions. The rare earth elements in the two or more rare earth-containing compositions can be the same or can differ. CeO₂ and Ce₂O₃ are a non-limiting example of differing rare earth-containing compositions having a common rare earth element. PrOz and CeO₂ are a non-limiting example of differing rare earth-containing compositions having differing rare earth elements.

In an embodiment of the present invention, the one or more rare earth-containing compositions comprise any one of:

a) one or more rare earths selected from the group of rare earths consisting essentially of cerium, lanthanum, and praseodymium;

b) no more than two rare earths selected from the group of rare earths consisting essentially of yttrium, scandium and europium. The rare earth composition can be sintered; and

c) one or more rare earths selected from the group of rare earths consisting essentially of yttrium, lanthanum, cerium praseodymium, scandium and europium.

The rare earths comprising the one or more rare earth-containing compositions can have oxidation states, valence states, or both that differ or are the same. Furthermore, the oxidation states, valance states, or both can have an integer or a fractional value. The rare earth-containing compositions may be available commercially, may be obtained from any source, or may be obtained through any process known to those skilled in the art.

Preferably, one or more rare earth-containing compositions is substantially insoluble in water under standard conditions of temperature and pressure. More specifically, one or more, or preferably all, of the one or more of the rare earth-containing compositions are substantially insoluble in water under standard conditions.

The disinfecting agent comprises rare earth-containing particulates present in the form of one or more of a granule, crystal, crystallite, particle or other particulate, referred to generally herein as a “particulate” or “particle.” The rare earth-containing particulates can comprise individual particles or an agglomeration or an aggregation of individual particles. As used herein, “particle” refers to a solid or microencapsulated liquid having a finite size, with no limitation in shape.

While not wanting to be bound by any theory, chemical reactivity and/or physical properties of a composition can be affected by particle or particulate size. More specifically, for relatively large-sized particles or particulates chemical and physical properties of the particles or particulates are substantially affected by compositional bulk properties. While for relatively small-sized particles or particulates the chemical and/or physical properties can differ from the large-sized particles or particulates. Large-sized particles or particulates have a smaller percentage of atoms per bulk of the particle or particulates than small-sized particles or particulates. The difference of one or both of the chemical and physical properties of the large- and small-sized particles or particulates can at least be due to an increase in the percentage of surface atoms in the small-sized particles or particulates compared to large-sized particles or particulates.

The rare earth-containing particles or particulates can have any shape, structure or size. The particles or particulates can resemble, without limitation, a spherical-shape, cylindrical-shape, a cube- or rectangular-shape. Moreover, the particles or particulates can have a plate-like, lamellar, a porous structure, or a combination thereof.

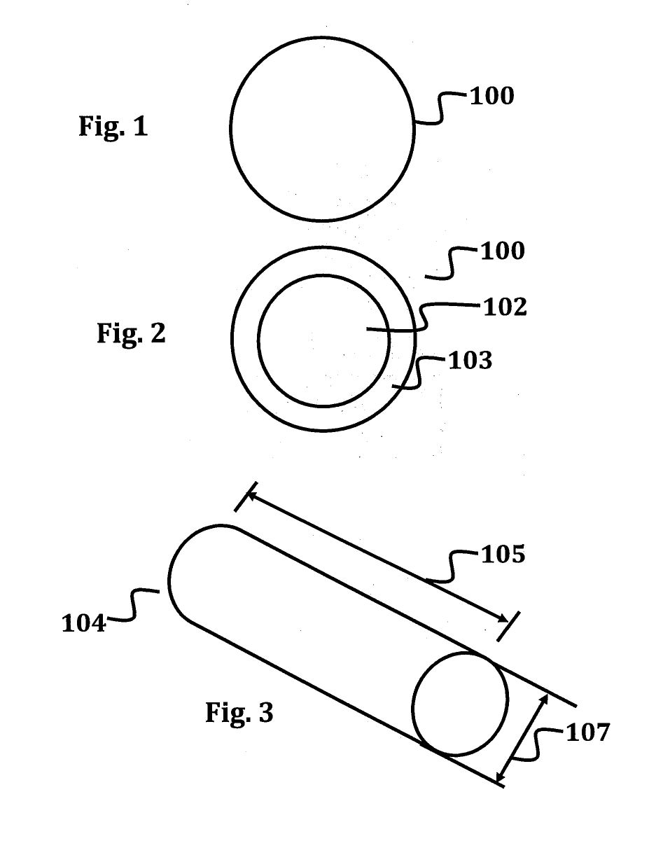

The rare earth-containing particles or particulates can have a shape substantially resembling a sphere 100 (FIG. 1). In some configurations, the sphere can be in the form a core-shell configuration 101 (FIG. 2) having a core 102 of one composition and a shell of another composition 103, the core 102 and shell 103 compositions differ in one or both of a chemical and physical property. In a preferred embodiment, the shell composition 103 comprises one or more rare earths and the core composition 102 comprises a composition substantially lacking rare earths. Preferred core compositions comprise non-rare earth minerals (such as, such clays, metal oxides, and metalloid oxides) and polymeric materials (including both, inorganic and organic polymeric materials).

The rare earth-containing particles or particulates can have a shape resembling a fiber 104 (FIG. 3), such as a cylindrical-shape having a cylinder-length 105 and a cylindrical-width 107. In one embodiment, the cylindrical-length 105 is at least greater than the cylindrical-width 107. In a preferred embodiment, the cylindrical-length 105 is at least about 1 times, more preferably at least about 2 times, even more preferably at least 3 times, even more preferably at least about 4 times, even more preferably at least about 5 times, even more preferably at least about 5 times, even more preferably at least about 6 times, even more preferably at least about 7 times, even more preferably at least about 8 times, even more preferably at least about 9 times, even more preferably at least about 10 times, even more preferably at least about 15 times, and even more preferably at least about 20 times the cylindrical-width 107. The rare-earth-containing fibers 104 can form fibrous structures, such as rare earth-containing filters.

In another embodiment of the present invention, the rare earth-containing fibers can be combined with a non-rare earth-containing material to form a fibrous substrate. The non-rare earth-containing material can be any material capable of being formed into a fiber. Non-limiting examples of such non-rare earth-containing materials are cellulosic materials, synthetic and/or natural polymers, metalloids, metals, and metal-containing materials. The rare earth-containing fibers and the non-rare earth-containing material can be held together largely by mechanical entrainment, that is, little, if any, binder is needed to hold together the rare earth-containing fibers and the non-rare earth-containing material. Preferably, the rare earth-containing fibers and the non-rare earth-containing material are held together substantially without another material, such as a binder material.

Non-limiting examples of cellulosic materials are: plant cell wall materials, cotton fibers, wood-based fibers, cellulose acetate, and rayon acetate. Non-limiting examples of synthetic polymers are: polyacetals, polyacrylics, polyanhydrides, polyamides, polycarbonates, polydienes, polyesters, polyhalo-olefins, polyimides, polyimines, polyketones, polyolefins, polyoxides, polyphylenes, polyphosphazenes, polysilanes, polysiloxanes, polystyrenes, polysulfides, polysulfoamides, polysulfonates, polysulfones, polysulfoxides, polythianhydrides, polythioamides, polythiocarbonates, polythioesters, polythioketones, polythioimides, polythioureas, polythiourethanes, polyureas, polyurethanes, polyvinyls, and mixtures thereof. Examples of natural polymers are plant, animal and mineral based polymers, such as, but not limited to, cellulosic polymers, chitin polymers, and protein based polymers (such as, silk, wool, leather, keratin).

Examples of metalloid, metal and metal-containing fibers can be any fiber comprising a metal or a metal-containing material. Examples of metals are any transition metal (that is, any metal contained with Groups 3-12, or Groups 1, 2, 13-17, 8, 1B or 2B of the NIST SP 966 Periodic Table) and any metalloid (such as, Al, Ga, Ge, In, Sn, Sb, Tl, Pb, Bi and Po). Examples of metalloid and/or metal-containing materials are any material containing a metalloid, metal, such as, a metal alloy, a compound comprising one or more metalloid, metal, and a substance and/or a composition containing one or more metalloid and/or metal (such as a natural or synthetic polymer coated with and/or compounded with at least one metal).

The rare earth-containing composition can be a water soluble composition, a water insoluble composition, a mixture of water soluble compositions, a mixture of water insoluble compositions, or a mixture of water soluble and insoluble compositions. Non-limiting examples of some insoluble rare earth compositions are rare earth oxides, fluorides, phosphates, oxy-chlorides, and carbonates. The rare earth compositions can be obtained from any source or through any process known to those skilled in the art.

More specifically depending upon the application, the water insoluble composition has a total preferred dissolved rare earth concentration of less than about 5×10⁻² M, of less than about 1×10⁻² M, more preferably of less than about 1×10⁻³ M, even more preferably of less than about 1×10⁻⁴ M, even more preferably of less than about 1×10⁻⁵ M, even more preferably of less than about 1×10⁻⁶ M, even more preferably of less than about 1×10⁻⁷ M, even more preferably of less than about 1×10⁻⁸ M, even more preferably of less than about 1×10⁻⁹ M, and even more preferably of less than about 1×10⁻¹⁰ M. In a preferred embodiment of the present invention, the water insoluble composition has a total dissolved cerium concentration preferably of less than about 5×10⁻² M, more preferably of less than about 1×10⁻² M, even more preferably of less than about 1×10⁻³ M, even more preferably of less than about 1×10⁻⁴ M, even more preferably of less than about 1×10⁻⁵ M, even more preferably of less than about 1×10⁻⁶ M, even more preferably of less than about 1×10⁻⁷ M, even more preferably of less than about 1×10⁻⁸ M, even more preferably of less than about 1×10⁻⁹ M, and even more preferably of less than about 1×10⁻¹⁰ M.

More specifically depending upon the application, the water soluble composition has a total dissolved rare earth concentration of preferably at least about 1 M, more preferably of at least about 1×10⁻¹ M, even more preferably of at least about 5×10⁻² M, even more preferably of at least about 1×10⁻² M, and even more preferably of at least about 1×10⁻³ M. In a preferred embodiment of the present invention, the water soluble composition has a total dissolved cerium concentration of preferably at least about 1 M, more preferably of at least about 1×10⁻¹ M, even more preferably of at least about 5×10⁻² M, even more preferably of at least about 1×10⁻² M, and even more preferably of at least about 1×10⁻³ M.

The insoluble rare earth-containing composition can comprise cerium and one or more of lanthanum, praseodymium, yttrium, scandium, and europium. Preferably, the total rare earth content of the rare earth-containing composition is at least about 75 wt %, more preferably at least about 80 wt %, even more preferably at least about 85 wt %, even more preferably at least about 90 wt %, even more preferably at least about 92 wt %, even more preferably at least about 94 wt %, even more preferably at least about 96 wt %, even more preferably at least about 98 wt %, even more preferably at least about 99 wt %, even more preferably at least about 99.9 wt %, even more preferably at least about 99.99 wt %, even more preferably at least about 99.999 wt %, and even more preferably at least about 99.9999 wt %. More preferably, the cerium earth content of the rare earth-containing composition is at least about 75 wt %, more preferably at least about 80 wt %, even more preferably at least about 85 wt %, even more preferably at least about 90 wt %, even more preferably at least about 92 wt %, even more preferably at least about 94 wt %, even more preferably at least about 96 wt %, even more preferably at least about 98 wt %, even more preferably at least about 99 wt %, even more preferably at least about 99.9 wt %, even more preferably at least about 99.99 wt %, even more preferably at least about 99.999 wt %, and even more preferably at least about 99.9999 wt %.

In a preferred embodiment of the present invention, the insoluble rare earth-containing composition comprises cerium and one or more of lanthanum, neodymium, praseodymium, and samarium. In another embodiment of the present invention, the one or more insoluble rare earth-containing composition comprises one or more of cerium, yttrium, scandium, and europium.

Furthermore, the insoluble rare earth-containing composition preferably has no more than about 10 wt % La, more preferably no more than about 9 wt % La, even more preferably no more than about 8 wt % La, even more preferably no more than about 7 wt % La, even more preferably no more than about 6 wt % La, even more preferably no more than about 5 wt % La, even more preferably no more than about 4 wt % La, even more preferably no more than about 3 wt % La, even more preferably no more than about 2 wt % La, even more preferably no more than about 1 wt % La, even more preferably no more than about 0.5 wt % La, and even more preferably no more than about 0.1 wt % La. The insoluble rare earth-containing composition preferably has no more than about 8 wt % Nd, more preferably no more than about 7 wt % Nd, even more preferably no more than about 6 wt % Nd, even more preferably no more than about 5 wt % Nd, even more preferably no more than about 4 wt % Nd, even more preferably no more than about 3 wt % Nd, even more preferably no more than about 2 wt % Nd, even more preferably no more than about 1 wt % Nd, even more preferably no more than about 0.5 wt % Nd, and even more preferably no more than about 0.1 wt % Nd. The insoluble rare earth-containing composition preferably has no more than about 5 wt % Pr, more preferably no more than about 4 wt % Pr, even more preferably no more than about 3 wt % Pr, even more preferably no more than about 2.5 wt % Pr, even more preferably no more than about 2.0 wt % Pr, even more preferably no more than about 1.5 wt % Pr, even more preferably no more than about 1.0 wt % Pr, even more preferably no more than about 0.5 wt % Pr, even more preferably no more than about 0.4 wt % Pr, even more preferably no more than about 0.3 wt % Pr, even more preferably no more than about 0.2 wt % Pr, and even more preferably no more than about 0.1 wt % Pr. The insoluble rare earth-containing composition preferably has no more than about 3 wt % Sm, more preferably no more than about 2.5 wt % Sm, even more preferably no more than about 2.0 wt % Sm, even more preferably no more than about 1.5 wt % Sm, even more preferably no more than about 1.0 wt % Sm, even more preferably no more than about 0.5 wt % Sm, even more preferably no more than about 0.4 wt % Sm, even more preferably no more than about 0.3 wt % Sm, even more preferably no more than about 0.2 wt % Sm, even more preferably no more than about 0.1 wt % Sm, even more preferably no more than about 0.05 wt % Sm, and even more preferably no more than about 0.01 wt % Sm.

When the rare earth composition comprises a cerium-containing compound, the cerium-containing compound can be derived from an organic and inorganic cerium-containing compound. More specifically, the cerium-containing compound may be derived from one or more of a cerium carboxylic acid salt (such as without limitation, cerium formate, cerium acetate, cerium oxalate, cerium fumarate, cerium gultamate, or cerium glutarate) or one or more of cerium carbonate, cerium nitrate, cerium hydroxide, cerium borate, cerium phosphate, cerium halides, a cerium-salt of a mineral acid, and/or a cerium compound formed by a precipitation process. Preferably, the insoluble rare earth-containing composition is derived from a thermal decomposition process, cerium oxide being one, non-limiting example of, an insoluble rare earth-containing composition formed by a thermal decomposition process. The insoluble rare earth composition can be a cerium oxide or a mixture of cerium (III) and (IV) oxides, and optionally, one or more of a binder and/or a support (such as but not limited to a polymeric or a natural fiber binder or a metal, mineral and/or metalloid support).

The rare earth-containing composition may be a sintered rare earth-containing composition. In one embodiment, the sintered rare earth-containing composition includes no more than two elements selected from the group of rare earths consisting of yttrium, scandium and europium.

The rare earth-containing particles can comprise rare earth-containing crystallites. As used herein, “crystalline” refers to a solid material having atoms, molecules, and/or ions in an orderly arrangement, such as in a repeating pattern, preferably the orderly arrangement is in each of the three spatial dimensions and defined by a crystallographic point group. The crystallite can comprise a single crystal or single-domain crystal. Preferably, at least most, if not all, of the rare earth-containing crystallite comprises a continuous crystal lattice. Preferably, the continuous crystal lattice substantially lacks any grain boundaries. While not wishing to be bound by any theory, it is believed that the grain boundaries can affect the physical and/or chemical properties of the rare earth-containing crystallite.

Furthermore, the rare earth-containing particles or particulates can comprise one or both of polycrystalline and paracrystalline phases. As used herein, “polycrystalline” refers to crystallites of differing sizes arranged in varying orientations. As used herein, “paracrystalline” refers to short and/or medium range crystalline ordering, such a lack of long-range ordering in at least one of the three spatial dimensions. Moreover, the rare earth-containing particles can comprise a plurality of crystallites in the form of a cluster.

The rare earth-containing particles or particulates can be made by any method and/or process. The particles or particulates can be formed by formed by any grinding, precipitating, calcining, thermal decomposition, and/or sintering process.

For example, the rare earth-containing particles agent may be derived from precipitation of a rare earth metal salt or from thermal decomposition of, for example, a rare earth metal carbonate, nitrate, oxalate or any of the other cerium-containing salts indicated above at a temperature preferably between about 100 to about 700° C. and even more preferably between about 180 and 350° C. in a furnace in the presence of an oxidant, such as air. Formation of the insoluble fixing agent is further discussed in co-pending U.S. application Ser. No. 11/932,837, filed Oct. 31, 2007, which is incorporated herein by this reference.

Another embodiment of the present invention is a process for forming rare earth-containing particles comprising autoclaving a rare earth composition to form an autoclaved rare earth composition and calcining the autoclaved rare earth composition. The rare earth-containing particles may be derived any rare earth salt. Preferably, the rare earth salt is a rare earth carbonate, nitrate, sulfate, borate, hydroxide, phosphate, halide, or any other mineral acid salt, oxalate, acetate, or other carboxylic acid salt, or anionic halogen oxide (such as, XO₃⁻, where X is one of chlorine, bromine or iodine). More preferably, the rare earth salt comprises a rare earth carbonate, such as cerium carbonate.

A suspension comprising the rare earth salt is formed and charged to an autoclave. The suspension may comprise any solvent capable of forming a suspension of a rare earth salt in the solvent. Preferably, the suspension is an aqueous suspension. The rare earth salt and the solvent may be combined in any ratio. The rare earth salt-to-solvent ratio is preferably from about 1:100 to 1:0.1, more preferably from about 1:20 to about 1:2, and even more preferably from about 1:8 to about 1:4 on a mass ratio basis. In a preferred embodiment, the mass ratio is preferably about 1:6.

After charging the rare earth-containing suspension to the autoclave, the autoclave is sealed and heated, under superatmospheric pressure, to form an autoclaved rare earth salt. The autoclave may be a lined or an unlined autoclave. Preferably, the autoclave is a stainless steel autoclave, such as a 316 stainless autoclave. Ideally, the autoclave is fitted with a burst disc. The pressure rating of the burst disc may be from about 100 psig to about 27,000 psig, more preferably from about 1,000 psig to about 5,000 psig.

The autoclave may be heated by any autoclave heating method known within the art. Suitable heating methods are oil heating, hot air heating, steam heating, electrical heating, resistance heating, and magnetic heating.

A gas may be charged to the autoclave to pressurize autoclave. The gas may be an inert gas or reactive gas. Non-limiting examples of suitable inert gases are nitrogen, helium, and argon. A non-limiting example of a reactive gas is oxygen. It can be appreciated that the heat applied to the sealed autoclave may further pressurize the autoclave. In a preferred embodiment, at least most, if not all, of the pressure applied during the autoclaving process is due to the applied heat.

Preferably, the suspension is heated in the sealed autoclave to a suspension temperature preferably of from about 50° C. to about 750° C., a more preferred suspension temperature of from about 100° C. to about 400° C., and even more preferred suspension temperature of about 200° C. The suspension temperature is maintained preferably for a time period of about 0.2 hours to about 48 hours, more preferably for a period of about 1 hour to about 8 hours, and even more preferably a period of about 2 hours. The autoclave pressure is maintained below the burst disc burst rating. Preferably, the autoclave pressure is maintained below about 5,000 psig, or more preferably below about 2,000 psig.

The suspension may be maintained in a substantially quiescent state during the autoclaving process. Preferably, the suspension is maintained substantially quiescent during the applying of one or both of the heat and pressure. Optionally, the method may further comprise, agitating the suspension during the applying of one or both of the heat and pressure. The agitation may be applied continuously or intermittently during the autoclaving process. The agitating may comprise, without limitation shaking, stirring, circulating, shearing, high velocity shearing, rocking, tilting, or rotating of the autoclave, and/or purging (with a gas or other fluid) the suspension.

The method may further comprise applying ultrasonic energy to the suspension. The ultrasonic energy may be applied during at least some or all of the period of time of applying one or both of the heat and pressure to the suspension. Furthermore, the ultrasonic energy may be applied to the suspension prior to and/or after application of one or both of the heat and pressure to the suspension. Preferably, the ultrasonic energy is applied after the applying of heat and pressure to suspension.

After the applying of one or both of heat and pressure to the suspension in the sealed autoclave, the autoclave contains an autoclaved suspension comprising an autoclaved rare earth salt. Some or all of the autoclaved suspension is removed from the autoclave. The autoclaved suspension may be or may not be cooled before being removed from the autoclave.

In one embodiment of the present invention, all of the autoclaved suspension is removed from the autoclave and dried. The autoclaved suspension is dried at a temperature of preferably from about 10 to about 200 degrees Celsius, more preferably of from about 20 to about 150 degrees Celsius, even more preferably of from about 20 to about 100 degrees Celsius, and even more preferably of from about 30 to about 80 degrees Celsius. Preferably, the autoclaved suspension is dried at a temperature of no more than about 300 degrees Celsius, more preferably of no more than about 250 degrees Celsius, even more preferably of no more than about 200 degrees Celsius, even more preferably of no more than about 150 degrees Celsius, even more preferably of no more than about 100 degrees Celsius, even more preferably of no more than about 80 degrees Celsius, even more preferably of no more than about 70 degrees Celsius, and even more preferably of no more than about 50 degrees Celsius. After drying, the dried suspension is calcinated.

In another embodiment of the present invention, the autoclaved suspension comprises a substantially liquid phase and a substantially solid phase. The substantially liquid phase comprises autoclaved rare earth salt suspended in the solvent. The solid phase comprises a precipitated and autoclaved rare earth salt; that is, the solid phase contains the rare earth salt that has precipitated and/or settled out from the solvent during the autoclaving process. The liquid and solid phases are removed separately from the autoclave. One or both of the liquid and solid phases are dried and calcinated. The liquid and solid phases are dried as described about for the autoclaved suspension. The dried liquid phase substantially comprises the suspended autoclaved rare earth salt. The dried solid phase substantially comprises the precipitated autoclaved rare earth salt.

The calcining process comprises heating the one or more of the dried autoclaved suspension, the dried liquid phase, or dried solid phase in a furnace to a preferred temperature of from about 200 degrees Celsius to about 500 degrees Celsius, more preferably of from about 250 degrees Celsius to about 350 degrees Celsius, and even more preferably at about 300 degrees Celsius to form rare earth-containing particles. The furnace can comprise any furnace capable of achieving any of the indicated temperatures. Preferably, the furnace is a muffle furnace.

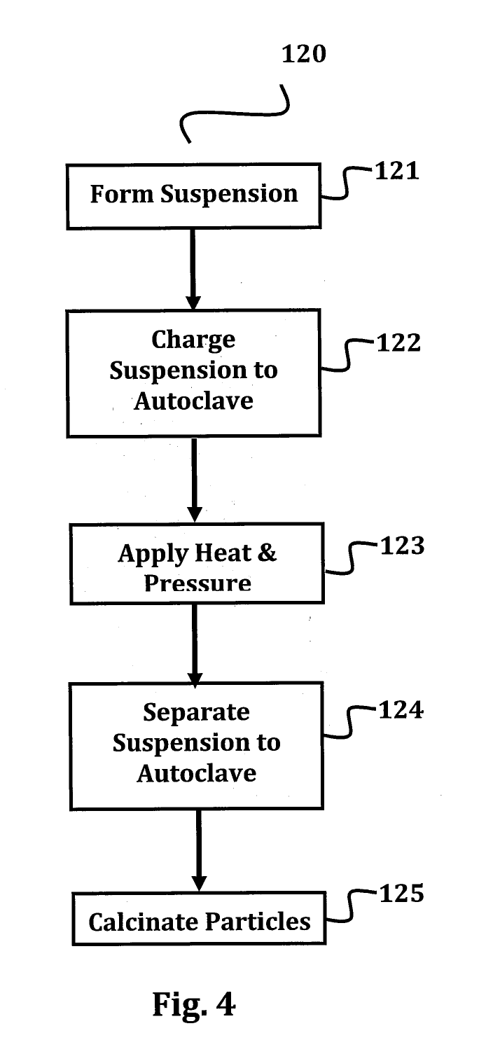

FIG. 4 depicts a first method 120 for making rare earth-containing particles, comprising:

(a) forming a suspension of a rare earth salt (step 121);

(b) charging the suspension to an autoclave (step 122);

(c) applying one or both of heat and superatmospheric pressure to the suspension to form an autoclaved suspension (step 123);

(f) separating the autoclaved suspension into a liquid phase and a solid phase (step 124); and

(g) calcining the liquid phase to form rare earth-containing particles (step 125). Preferably, the suspension comprises an aqueous suspension. Moreover, the rare earth salt is preferably a substantially insoluble rare earth salt. Preferably, the autoclave is sealed prior the application of one or both of heat and pressure to the suspension. In a preferred embodiment, the suspension is substantially quiescent during the application of the one or both of heat and pressure to the suspension. The separating of the autoclaved suspension into a liquid phase and a solid phase may be any known separation process, such as, decantation, piping and/or suctioning off the liquid layer, filtration, or a combination thereof. Optionally, the liquid phase is dried prior to calcining. The liquid phase may be dried by any drying process, such as, but not limited to, air drying, vacuum drying, drying at an above ambient temperature by applying heat, or a combination thereof.

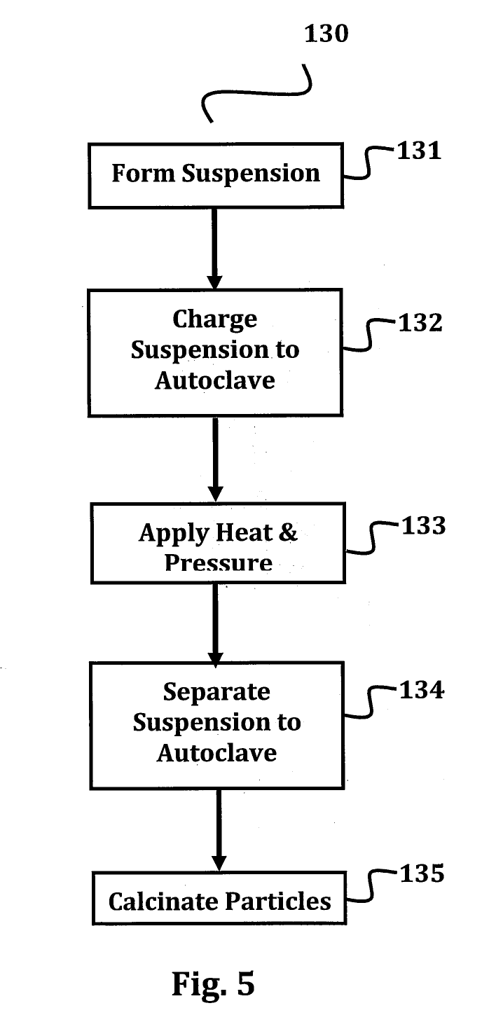

FIG. 5 depicts a second method for 130 for making rare earth-containing particles, comprising:

(a) forming a suspension of a rare earth salt (step 131);

(b) charging the suspension to an autoclave (step 132);

(c) applying one or both of heat and superatmospheric pressure to the suspension to form an autoclaved suspension (step 133);

(d) separating the autoclaved suspension into a liquid phase and solid phase (step 134); and

(e) calcining the solid phase to form rare earth-containing particles (step 135). Preferably, the suspension comprises an aqueous suspension. Preferably, the rare earth salt is a substantially insoluble rare earth salt. Commonly, the autoclave is sealed prior the the application of one or both of heat and pressure to the suspension. In a preferred embodiment, the suspension is substantially quiescent during the application of the one or both of heat and pressure to the suspension. The separating of the autoclaved suspension into a liquid phase and a solid phase may be any known separation process, such as, decantation, piping and/or suctioning off the liquid layer, filtration, or a combination thereof. The solid phase may be dried by any drying process, such as, but, not limited to, air drying, vacuum drying, drying at an above ambient temperature by applying heat, washing the solid with a drying solvent, or a combination thereof.

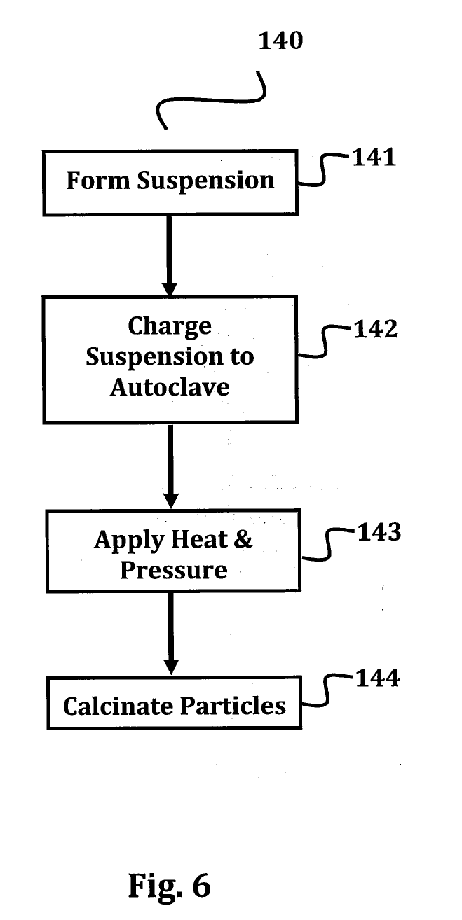

FIG. 6 depicts a second method for 140 for making rare earth-containing particles or particulates, comprising:

(a) forming a suspension of a rare earth salt (step 141);

(b) charging the suspension to an autoclave (step 142);

(c) applying one or both of heat and superatmospheric pressure to the suspension to form an autoclaved suspension (step 143); and

(d) calcining the solid phase to form rare earth-containing particles (step 144). Preferably, the suspension comprises an aqueous suspension. Moreover, the rare earth salt is preferably a substantially insoluble rare earth salt. Preferably, the autoclave is sealed prior to application of one or both of heat and pressure to the suspension. In a preferred embodiment, the suspension is substantially quiescent during the application of the one or both of heat and pressure to the suspension. Optionally, the autoclaved suspension may be removed from the autoclave before calcining. Furthermore, the autoclaved suspension may be optionally dried prior to calcining.

The rare earth-containing particle size can vary depending upon one or both of the method of preparation and the method of use of the rare earth-containing particles. While not wanting to be limited by example, small-size particles or particulates are preferred for spray and cream formulations, while large-size particles or particulates are preferred for supported particle applications. In one embodiment, the average particle or particulates size is preferably less than about 1,000 microns, more preferably less than about 500 microns, even more preferably less than about 200 microns, even more preferably less than about 100 microns, even more preferably less than about 70 microns, even more preferably less than about 30 microns, even more preferably less than about 20 microns, even more preferably less than about 10 microns, even more preferably less than about 5 microns, even more preferably less than about 1 micron, even more preferably less than about 500 nanometers, even more preferably less than about 100 nanometers, even more preferably less than about 50 nanometers, even more preferably less than about 20 nanometers, even more preferably less than about 10 nanometers, even more preferably less than about 5 nanometers, and even more preferably less than about 1 nanometer. In another embodiment, the average particle or particulate size is preferably one of: from about 1,000 microns, from about 500 microns, from about 200 microns, from about 100 microns, from about 70 microns, from about 30 microns, from about 20 microns, from about 10 microns, from about 5 microns, from about 1 micron, from about 500 nanometers, from about 100 nanometers, from about 50 nanometers, from about 20 nanometers, from about 10 nanometers, from about 5 nanometer, from about, or from about nanometers, to one of: of about 1,000 microns, of about 500 microns, of about 200 microns, of about 100 microns, of about 70 microns, of about 30 microns, of about 20 microns, of about 10 microns, of about 5 microns, of about 1 micron, of about 500 nanometers, of about 100 nanometers, of about 50 nanometers, of about 20 nanometers, of about 10 nanometers, of about 5 nanometer, of about, of about 1 nanometer, or of about 0.1 nanometers.

In one embodiment, the rare earth-containing particles or particulates have a mean diameter. The mean diameter can be expressed is in terms of one or more of the following: MV, MN and MA. MV is the mean diameter of the volume distribution and represents the center of gravity of the distribution. The mean volume diameter is weighted (that is, strongly influenced) by any change in the volume amount of larger particles or particulates in particle or particulate distribution. MN is the mean diameter of the number distribution and is calculated using the volume distribution and is weighted to the smaller particles or particulates in the distribution. MA is the mean diameter of the area distribution and is calculated from the volume distribution. The mean area diameter is less weighted (that is, less sensitive) than the mean volume diameter to changes in the amount of large particles or particulates in the distribution. The mean area diameter also represents information about the surface area of the particles or particulates. The mean volume, mean number and mean area diameters are calculated as follows:

MV=ΣVᵢdᵢ/ΣVᵢ (1)

MN=Σ(Vᵢdᵢ²)/ΣVᵢdᵢ³) (2)

MA=ΣVᵢ/Σ(Vᵢ/dᵢ) (3)

where, Vᵢ is volume percent of each size center i, and dᵢ is particle or particulate size for each size center i.

Rare earth-containing particle or particulate size distributions according to various embodiments of the present invention are depicted in FIGS. 6-13. The rare earth-containing particle or particulate size range and/or distribution depicted in FIGS. 6-13 are illustrative and non-limiting to the rare earth-containing particle or particulate size ranges and/or distributions enabled by the present disclosure.

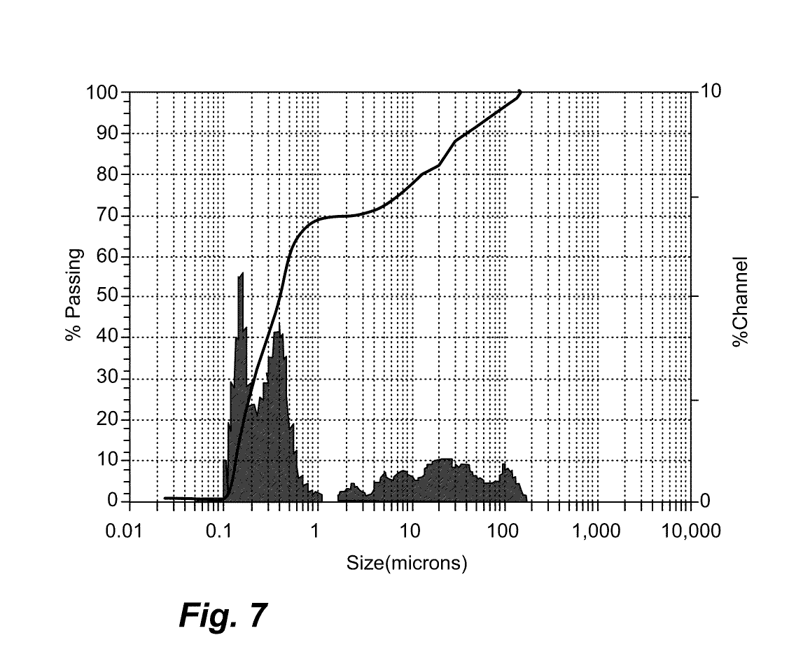

FIG. 7 depicts a mean particle or particulate size volume distribution (MV) for the rare earth-containing particles or particulate according to a first particle or particulate size embodiment of the present invention. The mean particle or particulate size distribution is bimodal. Preferably, at least most of the particles or particulates have a particle or particulate size from about 0.1 microns to about 1 micron. More preferably, at least about 70% of the particles or particulates have a mean particle diameter from about 0.1 microns to about 1 micron. Preferably at most about 30% of the particles or particulates have a mean particle diameter from about 2 microns to about 200 microns. The mean particle or particulate size is preferably about 12 microns. The standard deviation for the distribution is preferably about 11. The mean particle or particulate size for a number distribution of particle or particulate size is preferably about 0.2 microns. The mean particle or particulate size for a surface distribution is preferably about 0.3 microns.

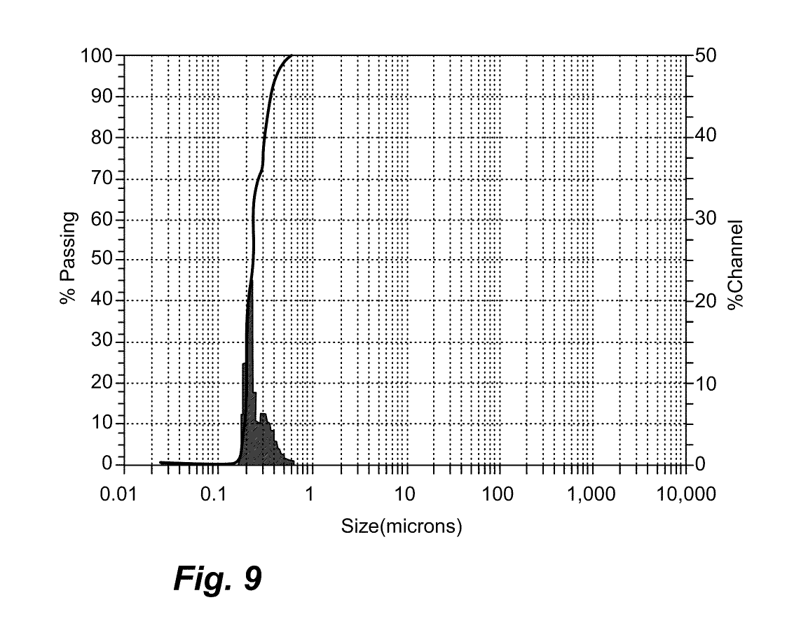

FIG. 9 depicts a mean particle size volume distribution (MV) for the rare earth-containing particles or particulates according to a second particle or particulate size embodiment of the present invention. The mean particle or particulate size distribution depicted is a broad, multi-modal particle or particulate size distribution. The particle or particulate size distribution preferably has large a standard deviation of about 183. Preferably, at least about 10% of the particles or particulates have a particle or particulate size of from about 0.2 microns to about 7 microns. More preferably, about 40% of particles or particulate have a particle or particulate size from about 7 microns to about 300 microns and even more preferably about 50% of the particles or particulates have a particle or particulate size from about 300 to about 500 microns. The average particle or particulate size is preferably about 223 microns. The mean particle or particulate size for a number distribution of particle or particulate size is preferably about 0.4 microns. The mean particle or particulate size for a surface distribution is preferably about 5 microns.

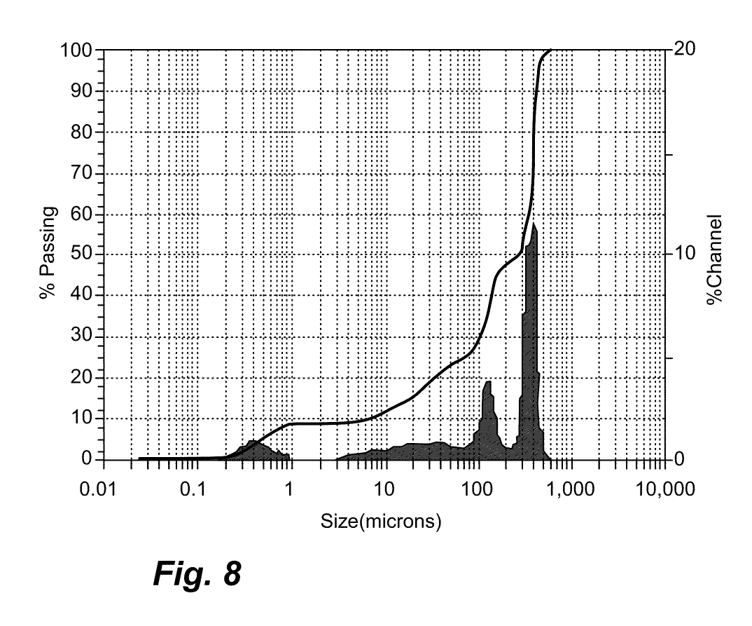

FIG. 8 depicts a mean particle or particulate size volume distribution (MV) for the rare earth-containing particles or particulates according to a third particle or particulate size embodiment of the present invention. The mean particle or particulate size distribution depicted is a narrow particle or particulate size distribution preferably having a standard deviation of about 0.07. At least about 90% of the particles or particulates preferably have a particle or particulate size of from about 0.2 microns to about 0.4 microns. About 100% of particles or particulates preferably have a particle or particulate size from about 0.2 microns to about 0.7 microns. The average particle or particulate size is preferably about 0.25 microns. The mean particle or particulate size for a number distribution of particle or particulate size is preferably about 0.22 microns. The mean particle or particulate size for a surface distribution is preferably about 0.25 microns.

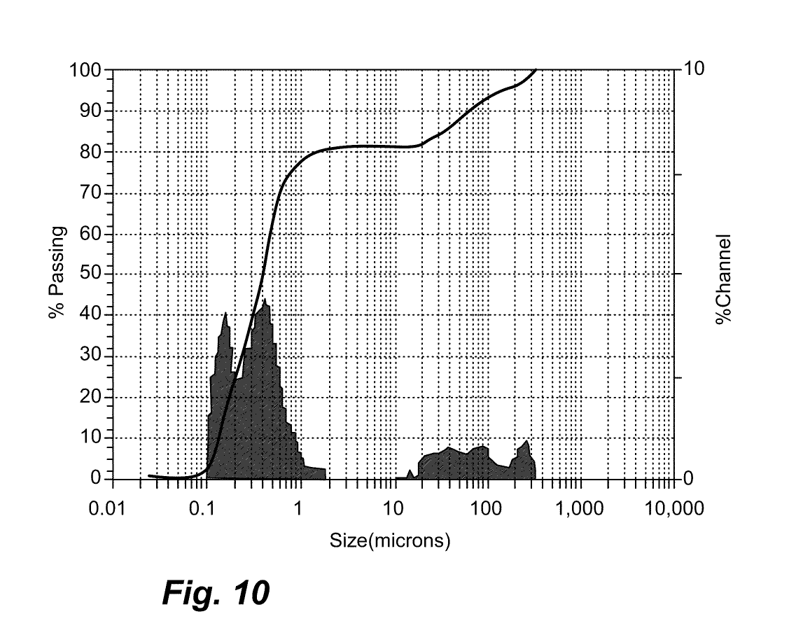

FIG. 10 depicts a mean particle or particulate size volume distribution (MV) for the rare earth-containing particles or particulates according to a fourth particle or particulate size embodiment of the present invention. The mean particle or particulate size distribution depicted is a narrow particle or particulate size distribution having a standard deviation preferably of about 15. At least about 80% of the particles or particulates preferably have a particle or particulate size of from about 0.1 microns to about 2 microns. About 100% of particles or particulates preferably have a particle or particulate size of from about 0.1 microns to about 300 microns. The average particle or particulate size is preferably about 20 microns. The mean particle or particulate size for a number distribution of particle or particulate size is preferably about 0.15 microns. The mean particle or particulate size for a surface distribution is preferably about 0.3 microns.

In a fifth particle or particulate size embodiment of the present invention the distribution is substantially broad particle or particulate size distribution. At least about 100% of the particles or particulate have a preferred particle or particulate size from about 0.3 microns to about 500 microns. The average particle or particulate size is about 95 microns. The particle or particulate size distribution has a preferred standard deviation of about 85. The mean particle or particulate size for a number distribution of particle or particulate size is preferably about 0.4 microns. The mean particle or particulate size for a surface distribution is preferably about 20 microns.

In a sixth particle or particulate size embodiment of the present invention, at least most of the rare earth-containing particles or particulates have a preferred mean particle or particulate diameter between about 1 to about 10 nanometers. Preferably at least about 75 wt %, more preferably at least about 85 wt %, even more preferably at least about 90 wt %, and even more preferably at least 98 wt % of the rare earth-containing particles or particulates have a mean particle or particulate diameter between about 1 to about 10 nanometers.

In a seventh particle or particulate size embodiment of the present invention, at least most of the rare earth-containing particles or particulates have a preferred mean diameter between about 0.1 to about 1 nanometer. Preferably, at least about 75 wt %, more preferably at least about 85 wt %, even more preferably at least about 90 wt %, and even more preferably at least 98 wt % of the rare earth-containing particles or particulates have a mean diameter between about 0.1 to about 1 nanometer.

In another particle or particulate size embodiment of the present invention, the graphical standard deviation of the rare earth-containing particle or particulate size distribution is preferably no more than about 250, more preferably no more than about 200, even more preferably no more than about 150, even more preferably no more than about 100, even more preferably no more than about 50, even more preferably no more than about 25, even more preferably no more than about 10, even more preferably no more than 4, even more preferably no more than about 2, even more preferably no more than about 1, even more preferably no more than 0.7, even more preferably no more than about 0.5, even more preferably no more than about 0.3, and even more preferably no more than 0.1. Preferably, the graphical standard deviation of the rare earth-containing particle or particulate size distribution is no more than about 25, more preferably no more than about 10, even more preferably no more than 4, even more preferably no more than about 2, even more preferably no more than about 1, even more preferably no more than 0.7, even more preferably no more than about 0.5, even more preferably no more than about 0.3, and even more preferably no more than 0.1.

The rare earth-containing particles or particulates preferably have a mean surface area per unit mass of at least about 1 m²/g. More preferably, the rare earth-containing particle or particulate has a surface area per unit mass of at least about 5 m²/g, more preferably of at least about 10 m²/g, even more preferably of at least about 100 m²/g, even more preferably of at least about 150 m²/g, even more preferably of at least about 300 m²/g, and even more preferably of at least about 400 m²/g. Even more preferably, the rare earth-containing particle or particulate has any average particle or particulate size and any particle or particulate distribution and a surface area per unit mass of at least about 1 m²/g, even more preferably of at least about 5 m²/g, even more preferably of at least about 10 m²/g, even more preferably of at least about 100 m²/g, even more preferably of at least about 150 m²/g, even more preferably of at least about 300 m²/g, and even more preferably of at least about 400 m²/g.

### Contacting of the Disinfecting Agent with the Infectious Matter

Contacting the disinfecting agent with the infectious matter one or both of deactivates and kills the infectious matter. In one embodiment, the infectious matter chemically, physically or both chemically and physically interacts with the disinfecting agent when contacted with the disinfecting agent. That is, contacting the infectious matter with the disinfecting agent chemically and/or physically changes the infectious matter. The chemical and/or physical change can be a chemical reaction, a physical change, a chemical degradation, a physical damage, or any combination thereof at least one or more vital entities of the infectious matter.

The infectious matter has a pre-contacting infectious matter population. Contacting the disinfecting agent with the infectious matter deactivates at least some, if not at least most or all, of the infectious matter to form a post-contacting infectious matter population. As used herein, “deactivates” refers to killing, damaging, or both killing and damaging the infectious matter to at least inhibit, if not stop, the infectious matter from one or both of causing disease and infection and from further reproduction. In one particular embodiment, it is believed that the contacting of the disinfecting agent with the infectious matter chemically and/or physically sufficiently damages and/or disrupts the cellular structure of the bacteria, fungi, or protozoa or the membranous envelope of the virus to kill and/or deactivate the infectious matter. Preferably, the post-contacting infectious matter population is at least less than the pre-contacting infectious matter population. The post-contacting infectious matter population divided by the pre-contacting infectious matter population forms a deactivation quotient. Preferably, the deactivation quotient is less than 1, more preferably is no more than about 10⁻¹ times more, even more preferably is no more than about 10⁻² times more, even more preferably is no more than about 10⁻³ times more, even more preferably is no more than about 10⁻⁴ times more, even more preferably is no more than about 10⁻⁵, even more preferably is no more than about 10⁻⁶ times more, even more preferably is no more than about 10⁻⁷, even more preferably is at least 10⁻⁸ times more, even more preferably is no more than about 10⁻⁹ times more, and even more preferably is no more than about 10⁻¹⁰.

In a virus, the vital entity can comprise genetic material (such as DNA or RNA), a protein material (such as, protein material protecting the genetic material within the virus), a lipid material (which surrounds or coats the protein material in some viruses), or a combination thereof. In a prokaryotic cell, such as a bacterium, the vital entity can comprise: a) an outermost region of a cellular envelope (such as, without limitation flagella or pili); b) the cellular envelope (such as, a cell wall and/or capsule) which provides rigidity to the cell and separates the environment from the cellular interior; c) a cytoplasmic region (such as, cellular DNA, ribosomes, inclusions, chromosomes, and plasmids) contained within the cellular interior; or d) combinations thereof. In an eukaryotic cell wall of a fungus (including mold and mildew), plant, or animal the vital entity can comprise: a) an outermost cellular region (such as, cilia or flagella); b) a plasma membrane (which may or may not form a cellular wall) separating the environment from the cellular interior; c) a cellular nucleus (such as, eukaryotic DNA or chromosomes, histone proteins, mitochondria, c) contained within the cellular interior; or d) a combination thereof. In a prion the vital entity can comprise an aberrantly shaped or miss-folded protein.

The chemical and/or physical change caused by the disinfecting agent can be a sorption or interaction of the disinfecting agent with the infectious matter that kills, deactivates, or both kills and deactivates the infectious matter. While not wanting to be limited by theory it is believed that the sorption and/or interaction of the infectious matter with the disinfecting agent chemically, physically or both chemically and physically deactivates and/or kills the disease causing agent. Moreover, it is believed disinfecting agents having greater mean surface areas may be more effective in killing and/or deactivating the infectious matters on a per mass basis.

As used herein, “chemical impairment” refers to the infectious matter being chemically impaired or killed by the disinfecting agent. As used herein, “chemically” or “chemical” refers to any property becoming evident by a chemical changed achieved through a chemical reaction.

As used herein, “physical impairment” refers to infectious matter being physically imparted and/or killed by the disinfecting agent. As used herein “physically” or “physical” refers to any measurable property, typically in terms of a Newtonian property describing a system's state at any given time without changing the system's identity.

More specifically, chemical impairment, physical impairment, or a combination thereof of the infectious matter by the disinfecting agent can substantially: a) prevent prophylactically the infectious matter from inducing one or both of a disease and an infection; b) preclude the infectious matter from perpetuating one or both of a disease and an infection; c) disinfect a target zone, or d) a combination thereof.

As used herein, “adsorption” refers to the adherence of atoms, ions, molecules, polyatomic ions, or other substances of a gas or liquid to the surface of another substance, called the adsorbent. The attractive force for adsorption can be, for example, chemical, such any chemical bond formation process, or physical such any force including without limitation ionic, electrostatic, van der Waals and/or London forces.

As used herein, “absorption” refers to the penetration of one substance into the inner structure of another, as distinguished from adsorption. As used herein, “sorb” or “sorption” refers to adsorption and/or absorption.

### Devices Using the Disinfecting Agent

The disinfecting agent can be used in a plurality of differing devices. Preferably, the disinfecting agent is present in each of the devices in an effective therapeutic amount. As used herein, “an effective therapeutic amount” refers to an amount to sufficiently treatment to one or both kill and deactivate at least some of infectious matter.

### Textile and Method for Making the Same

One embodiment of the present invention comprises a textile containing the disinfecting agent and a method for making the same. The embodiment includes any textile item comprising woven or non-woven textile items containing the disinfecting agent. Furthermore, the textile items include textile fabrics containing the disinfecting agent and any item fabricated with a textile fabric containing the disinfecting agent. Non-limiting examples of non-apparel textile items include, without limitation, carpets, rugs, drapes, curtains, sheets, blankets, pillowcases, pillows, mattress covers, mattresses, underwear, socks, shoe cushions, shoe linings, towels, feminine hygiene products, baby diapers, laboratory coats, patient clothing, and slip covers.

While not wanting to be limited by any example, the disinfecting agent comprises preferably no more than about 0.01 wt % of the textile, more preferably no more than about 0.05 wt % of the textile, even more preferably no more than about 0.1 wt % of the textile, even more preferably no more than about 0.2 wt % of the textile, even more preferably no more than about 0.5 wt % of the textile, even more preferably no more than about 0.8 wt % of the textile, even more preferably no more than about 1 wt % of the textile, even more preferably no more than about 2 wt % of the textile, even more preferably no more than about 3 wt % of the textile, even more preferably no more than about 4 wt % of the textile, even more preferably no more than about 5 wt % of the textile, even more preferably no more than about 6 wt % of the textile, even more preferably no more than about 8 wt % of the textile, even more preferably no more than about 10 wt % of the textile, even more preferably no more than about 12 wt % of the textile, even more preferably no more than about 15 wt % of the textile, and even more preferably no more than about 20 wt % of the textile. When the disinfecting agent is positioned between sheets of textile fabric (such as in a pillow or quilting manner) the disinfecting agent comprises preferably at least about 0.1 wt % of the textile, more preferably at least about 0.2 wt % of the textile, even more preferably at least about 0.5 wt % of the textile, even more preferably at least about 0.8 wt % of the textile, even more preferably at least about 1 wt % of the textile, even more preferably at least about 2 wt % of the textile, even more preferably at least about 3 wt % of the textile, even more preferably at least about 4 wt % of the textile, even more preferably at least about 5 wt % of the textile, even more preferably at least about 6 wt % of the textile, even more preferably at least about 8 wt % of the textile, even more preferably at least about 10 wt % of the textile, even more preferably at least about 12 wt % of the textile, even more preferably at least about 15 wt % of the textile, and even more preferably at least about 20 wt % of the textile.

Preferably, the textile comprises cerium oxide in an effective therapeutic amount. More preferably, the textile comprises cerium oxide in an amount ranging from about 0.01 wt % to about 20 wt %, even more preferably from about 0.05 wt % to about 10 wt %, even more preferably from about 0.1 wt % to about 5 wt %. When the disinfecting agent is positioned agent between sheets of textile fabric (such as in a pillow or quilting manner) the disinfecting agent comprises preferably from about 0.1 wt % to about 99 wt % cerium oxide, more preferably from about 0.2 wt % to about 95 wt % cerium oxide, and even more preferably from about 1 wt % to about 90 wt % cerium oxide.

Methods for making a textile comprising the disinfecting agent include any method for incorporating and/or adhering the disinfecting agent onto and/or within the textile. For example, without limitation, the fibers and/or yarns comprising the textile can have the disinfecting agent incorporated within the fiber and/or yarn during formation, such as, during spinning or extrusion of a fiber (such as, melt, extrusion or solution spinning) or twisting or other bonding of fibers (such as, staple or tow fibers) into a yarn or thread. In another non-limiting example, the disinfecting agent can be adhered to the textile by one or more of thermal, adhesive, physical, and chemical processes. The thermal process can include embedding the disinfecting agent into a thermally softened textile and/or fiber. In the thermal process, the disinfecting agent can be in direct contact with the textile and/or fiber and substantially directly adhered to the textile and/or fiber. The adhesive process can include bonding the disinfecting agent to the textile and/or fiber with a third material, such as an adhesive and/or coating composition. The third material is positioned between the disinfecting agent and textile and/or fiber. The disinfecting agent is adhered to the textile and/or fiber by the third material. The physical process can include one or both of a mechanical entrapment and/or electrostatic adherence of the disinfecting agent. The mechanical entrapment can include: a) positioning the disinfecting agent between sheets of textile fabric (such as in a pillow or quilting manner); b) entrapping the disinfecting agent between the interlocking fibers forming a yarn; c) entrapping the disinfecting agent with the fibers and/or yarns forming the woven or non-woven textile fabric; d) or any combination thereof. The electrostatic adherence can include any electrostatic attraction of the disinfecting agent and the textile and/or the fibers comprising the textile. The chemical process can include any process that forms a chemical bond between the disinfecting agent and the textile material (including the fibers comprising the textile material).

Moreover, the disinfecting agent can be incorporated into the textile by forming a coating comprising the disinfecting agent. Preferably, a deposition and/or coating process forms the disinfecting agent coating on the textile. Non-limiting examples of suitable processes include sol gel processes, a chemical deposition or precipitating processes, a vapor deposition processes, binder and binder-less coating processes, electrochemical deposition processes, and thermal deposition processes. Preferably, the deposition and/or coating process substantially coats at least some, if not at least most or all, of the textile. The coating can be a continuously or a discontinuously distributed over the textile. Furthermore, the coating can have a substantially uniform or a substantially non-uniform in thickness.

A non-limiting example of textile comprising a disinfecting agent is an antimicrobial fiber having rare earth-containing particles.

A non-limiting example may comprise: preparing a rare earth -containing solution; contacting a fiber (such as a plant fiber) with the rare earth-containing solution (such as by soaking or spraying) to form a fiber impregnated with the rare earth-containing solution; drying the fiber impregnated with the rare earth-containing solution to form a fiber having rare-containing particles.

The rare earth-containing particles can have any average particle size and/or surface area described above. The amount of the rare earth-containing particles in the fiber is preferably up to about 0.05 wt %, more preferably up to about 0.1 wt %, even more preferably up to about 0.2wt %, even more preferably up to about 0.3 wt %, even more preferably up to about 0.4 wt %, even more preferably up to about 0.5wt %, even more preferably up to about 0.6 wt %, even more preferably up to about 0.7 wt %, even more preferably up to about 0.8 wt %, even more preferably up to about 0.9 wt %, even more preferably up to about 1.0 wt %, even more preferably up to about 1.2 wt %, even more preferably up to about 1.4 wt %, even more preferably up to about 1.5 wt %, even more preferably up to about 1.6 wt %, even more preferably up to about 1.8 wt %, even more preferably up to about 2 wt %, even more preferably up to about 3 wt %, even more preferably up to about 3.5 wt %, even more preferably up to about 4 wt %, even more preferably up to about 4.5 wt %, and even more preferably up to about 5.0 wt % of the fiber. In one embodiment of the present invention, the amount of the rare earth-containing particles in the fiber is from about 0.1 wt % to about 1.5 wt %.

The fibers can be any fibers. Preferably, the fibers are water absorbent fibers such as, but not limited to cotton, linen, cellulosic fibers. The fibers can be blended with either other water absorbent fiber or non-water absorbent fibers.

The rare earth-containing solution can be any rare earth-containing solution having preferably at least about 5 g/L, more preferably at least about 10 g/L, even more preferably at least about 25 g/L, even more preferably at least about 50 g/L, even more preferably at least about 100 g/L, even more preferably at least about 150 g/L, even more preferably at least about 200 g/L, even more preferably at least about 250 g/L, even more preferably at least about 300 g/L, even more preferably at least about 350 g/L, even more preferably at least about 400 g/L, even more preferably at least about 450 g/L, and even more preferably at least about 500 g/L of a rare earth-containing composition. Preferably, the rare earth-containing composition comprises one of cerium nitrate or cerium chloride.

The rare earth-containing solution may contain a reducing agent. Glucose and starch are non-limiting examples of suitable reducing agents.

The fiber impregnated with the rare earth-containing solution may be dried at any temperature. More specifically, the fiber impregnated with the rare earth-containing solution may be dried at a temperature of preferably at least at about 15 degrees Celsius, more preferably at least at about 25 degrees Celsius, even more preferably at least at about 50 degrees Celsius, even more preferably at least at about 100 degrees Celsius, even more preferably at least at about 120 degrees Celsius, even more preferably at least at about 140 degrees Celsius, even more preferably at least at about 150 degrees Celsius, even more preferably at least at about 175 degrees Celsius, even more preferably at least at about 200 degrees Celsius, even more preferably at least at about 225 degrees Celsius, even more preferably at least at about 250 degrees Celsius, even more preferably at least at about 275 degrees Celsius, even more preferably at least at about 300 degrees Celsius, even more preferably at least at about 350 degrees Celsius, even more preferably at least at about 400 degrees Celsius, even more preferably at least at about 450 degrees Celsius, even more preferably at least at about 500 degrees Celsius, even more preferably at least at about 550 degrees Celsius, even more preferably at least at about 600 degrees Celsius, even more preferably at least at about 650 degrees Celsius, and even more preferably at least at about 700 degrees Celsius, or any combination thereof. In one embodiment of the present invention, the fiber impregnated with the rare earth-containing solution is dried from a temperature of about 120 degrees Celsius to about 200 degrees Celsius.

The fiber impregnated with the rare earth-containing solution may be dried for any period of time. More specifically, the fiber impregnated with the rare earth-containing solution may be dried at one or more of the above temperatures preferably for about 20 minutes, more preferably for about 40 minutes, even more preferably for about 60 minutes, even more preferably for about 1.5 hours, even more preferably for about 2 hours, even more preferably for about 3.5 hours, even more preferably for about 4 hours, even more preferably for about 5 hours, even more preferably for about 6 hours, even more preferably for about 7 hours, even more preferably for about 8 hours, even more preferably for about 10 hours, even more preferably for about 12 hours, even more preferably for about 14 hours, even more preferably for about 16 hours, even more preferably for about 18 hours, even more preferably for about 20 hours, even more preferably for about 24 hours, even more preferably for about 32 hours, even more preferably for about 36 hours, even more preferably for about 40 hours, even more preferably for about 48 hours, even more preferably for about 36 hours, and even more preferably for about 72 hours. In one embodiment of the present invention, the fiber impregnated with the rare earth-containing solution is dried from about 40 to about 60 minutes. In a more preferred embodiment, the fiber impregnated with the rare earth-containing solution is dried at a temperature from about 120 degrees Celsius to about 200 degrees Celsius for a period of time from about 40 to about 60 minutes.

Another textile embodiment of the present invention can comprise a bi-component fiber having a core component and a sheath component containing a disinfecting agent comprising a rare earth-containing composition. The core and sheath component can comprise any polymeric material. Preferably, the core and sheath components comprise thermoplastic polymers. The core and sheath components can comprise the same polymeric material or differing polymeric materials. Preferably, the core and sheath components comprise one or more of polyethylene terephthalate (PET), poly 1,4 cyclohexylene dimethylene terephthalate (PCT), polyethylene (PE), PETG (PET modified with 1,4, cyclohexanedimthanol), polypropylene (PP), co-PET, amorphous PET, polycaprolactam (PCL), or polybutylene terephthalate (PBT). The core component can comprise from about 5 to about 95 wt % of fiber. The sheath containing the disinfecting agent component can comprise from about 95% to about 5 wt % of the fiber. Preferably, the core component can comprise about 5 wt %, more preferably about 10 wt %, even more preferably about 15 wt %, even more preferably about 20 wt %, even more preferably about 25 wt %, even more preferably about 30 wt %, even more preferably about 35 wt %, even more preferably about 40 wt %, even more preferably about 45 wt %, even more preferably about 50 wt %, even more preferably about 55 wt %, even more preferably about 60 wt %, even more preferably about 65 wt %, even more preferably about 70 wt %, even more preferably about 75 wt %, even more preferably about 80 wt %, even more preferably about 85 wt %, even more preferably about 90 wt %, or even more preferably about 95 wt % of the fiber. Preferably, the sheath containing the disinfecting agent component can comprise from about 95%, more preferably about 90 wt %, even more preferably about 85 wt %, even more preferably about 80 wt %, even more preferably about 75 wt %, even more preferably about 70 wt %, even more preferably about 65 wt %, even more preferably about 60 wt %, even more preferably about 55 wt %, even more preferably about 50 wt %, even more preferably about 45 wt %, even more preferably about 40 wt %, even more preferably about 35 wt %, even more preferably about 30 wt %, even more preferably about 25 wt %, even more preferably about 20 wt %, even more preferably about 15 wt %, even more preferably about 10 wt %, or even more preferably about 5 wt % of the fiber. One or both of the core and sheath components can include polymeric additives, such as, but not limited to UV stabilizers, fire retardant additives, pigments, hydrophilic additives, anti-stain additives, rheology modifiers, viscosity modifiers, lubricants, fillers, and combinations or mixtures thereof.

The sheath has a thickness. Preferably, the thickness of the sheath can be no more than about 5%, more preferably about 10%, even more preferably about 15%, even more preferably about 20%, even more preferably about 25%, even more preferably about 30%, even more preferably about 35%, even more preferably about 40%, even more preferably about 45%, even more preferably about 50%, even more preferably about 55%, even more preferably about 60%, even more preferably about 65%, even more preferably about 70%, even more preferably about 75%, even more preferably about 80%, or even more preferably about 85 wt % of the total fiber cross-section. It can be appreciated that the ability to retain the disinfecting agent in the fiber is related the average mean particle and/or particulate size of the disinfecting agent. More specifically, the sheath component thickness is about equal to the average mean particle and/or particulate size of the disinfecting agent.

The bi-component fiber may be formed by the use of pellets of the two different polymers or a direct polymer stream from the reactor of which the fiber is to be formed. Two extruders are used to form the bi-component fiber. One extruder forms the core and another extruder forms the sheath. Polymer pellets for forming the core component are feed to the extruder which forms the core component, where the pellets are melted and extruded through a nozzle by a screw. In a similar manner, the disinfecting agent and polymer pellets for forming the sheath component are feed to the extruder which forms the sheath components, where the polymer is melted and mixed with the disinfecting agent and the mixture is extruded through a nozzle by a screw and around the core component.

### Apparel and Method for Making the Same

One embodiment of the present invention comprises an item of apparel containing the disinfecting agent and a method for making the same. More specifically, the embodiment includes any item of apparel worn by an animal, including a human. Non-limiting examples such apparel items include without limitation, a facemask, a gown (including a medical gown), an apron (including a surgical apron), a scrub-suit, a cab, a hat, a hairnet, a shoe cover, a glove (including, sterile, examination, and regular), undergarments (including foundations and support garments), or a diaper containing the disinfecting agent.

When the item of apparel comprises a textile, the disinfecting agent can be incorporated into the apparel item as described above for a textile. When the item of apparel comprises a non-textile item, the disinfecting agent can be incorporated into the non-textile item by one any one of the thermal, adhesive, physical, and chemical processes described above. Furthermore, the disinfecting agent can be incorporated into the non-textile item during and/or after the formation the non-textile item as described above. For example, the disinfecting agent can be incorporated during extrusion and/or molding of the item. Moreover, the disinfecting agent can be incorporated into the apparel item by any deposition and/or coating process as described above. Preferably, the item of apparel comprises the disinfecting agent at one of the levels indicated above.

### Medical Device and Method for Making the Same

One embodiment of the present invention is a medical device, medical apparatus, element or component of a medical device or apparatus, or combination hereof and a method for making the same. Non-limiting examples of medical devices include sutures, gauzes (including gauze bandages and wraps), sponges (including surgical sponges and peanuts), medical swabs (including cotton, polyester, and foam), dressings (including occlusive and non-occlusive), medical drapes (including surgical drapes), bandages (including steri-strips, elastic, adhesive, with or without a dressing, and compressive and non-compressive). Non-limiting examples of medical apparatuses include staplers (includes skin, duct, and vascular staplers and linear and circular staplers), surgical instruments (such as but not limited to hemostats, forceps, retractors, scalpels), light-handle covers, medical tubing, medical mesh (such as hernia mesh), wound drains, a medical implant (such as, a heart valve, a stent, an artificial joint, an orthopedic device, a dental implant, a dental device), and wound vacs.

When the medical device comprises a textile, the disinfecting agent can be incorporated into the textile as described above and in any one or more of the levels indicated above. Examples of medical devices comprising textiles are, without limitation, gauzes, swabs, sponges, drapes, and dressings.

When the medical device comprises a polymeric material, the disinfecting agent can be incorporated into the medical device during and/or after the formation of medical device as described above and at one of the levels indicated above.

When the medical device comprises a metallic material, the disinfecting agent can, where appropriate, be incorporated into the medical device by any one of the methods indicated above or by an alloying process. For example, most or all of the depositional and/or coating process can be appropriately applied to both polymeric and metallic materials. When the process comprises an alloying process, one or more rare earths are added during the alloy forming process. The alloy can comprise any amount of the one or more rare earths. Preferably, the alloy comprises cerium, more preferably cerium in the form of an oxide. The one or more rare earths are present at any one or more of the effective therapeutic levels indicated above for the disinfecting agent.

### Therapeutic Formulation and Method for Making the Same

Another embodiment of the present invention includes a therapeutic formulation and a method for making the same. A therapeutic formulation includes any formulation comprising the disinfecting agent in an effective amount. Non-limiting examples of formulations include aerosol sprays, solvent-based sprays, water-based sprays, powders (such as, foot, body, and crop or plant powders), creams, ointments, salves, liniments, and gels (including body, disinfecting or sanitizing, wound-treatment, anti-bacterial, and anti-fungal for animals or plants), a medical solution, and wound irrigation systems.

Preferably, the aerosol spray and the powder comprises the disinfecting agent having average particle or particulate size ranging from about 0.1 to about 1 nanometer, more preferably from about 1 nanometer to about 0.1 micron, even more preferably from about 0.1 to about 1 micron, and even more preferably from about 0.1 to about 100 microns. Furthermore, the disinfecting particles or particulates preferably have an average particle or particulate size of at least about 1 nanometer, more preferably of at least about 10 nanometers, even more preferably of at least about 50 nanometers, even more preferably of at least about 0.1 microns, even more preferably of at least about 1 micron, even more preferably of at least about 10 microns, even more preferably of at least about 50 microns, even more preferably of at least about 70 microns, even more preferably of at least about 100 microns, and even more preferably of at least about 200 microns. The aerosol spray can be formed by any suitable method known within the art for dispersing a powder. The powder can comprise the disinfecting agent formulated with other powder additives. The other powder additives can include non-caking additives (that is, additives to maintain the disinfecting powder in a “flowable” form) or coating additives (that is, additives to aid in the coating and/or adhering the disinfecting agent on the target zone).

The disinfecting agent practices can be dispersed or suspended in any suitable solvent for application to the target zone. Preferably, the disinfecting agent particles are dispersed or suspended in an aqueous system. In another embodiment, the disinfecting agent is dissolved in a solvent. Preferably, the disinfecting agent is dissolved in water. The aqueous system comprising the disinfecting agent in a dispersed, suspended or dissolved form can comprise one or more surfactants (including without limitation anionic surfactants, cationic surfactants, non-ionic surfactants, or combinations and mixtures thereof), wetting agents, viscosity modifiers, buffering agents, and pH modifiers. The aqueous system can have any pH. Basic pH values ranging from about pH 9 to about pH 10 are preferred. However, the aqueous system can have an acidic pH value of from about pH 1 to about pH 6, neutral pH of about pH 7, or basic pH value of from about pH 8 to about pH 12.

The disinfecting agent can be dispersed, suspended or dissolved in any medical solution. Non-limiting examples of suit medical solutions include acetic acid otic solution (a solution comprising glacial acetic acid in a solvent, typically a non-aqueous solvent), aluminum acetate topical solution (a solution comprising aluminum subacetate, glacial acetic acid, typically applied topically to the skin as a wet dressing or used as a gargle or mouthwash), aluminum subacetate solution (a solution comprising aluminum sulfate, acetic acid, calcium carbonate, and water, typically applied topically as wet dressing), anisotonic solution, anticoagulant citrate dextrose solution (an aqueous solution comprising citric acid, sodium citrate, and dextrose), anticoagulant heparin solution (an aqueous solution comprising sodium heparin and sodium chloride), anticoagulant sodium citrate solution (an aqueous solution comprising sodium citrate), Benedict's solution (an aqueous solution comprising sodium citrate, sodium carbonate and cupric sulfate), cardioplegic solution (comprising an aqueous or blood-containing solution typically containing potassium), Dakin's solution (comprising sodium hypochlorite), iodine solution (an aqueous solution one or both of iodine and sodium iodide, preferably from about 1.5 to about 2.5 grams of iodine and from about 2.0 to about 3.0 grams of sodium iodide), lactated Ringer's solution (an aqueous solution comprising calcium chloride, potassium chloride, sodium chloride, and sodium lactate), Lugol's or stong iodine solution (an aqueous solution comprising iodine and potassium iodide, preferably from about 3 to about 7 grams of iodine and from about 8 to about 12 grams of potassium iodide, more preferably about 5 grams of iodine, 10 grams of potassium iodide, and about 85 grams of water), Monsel's solution (an aqueous solution comprising basic ferric sulfate, an astringent and a hemostatic agent), normal saline solution (an aqueous solution comprising sodium chloride, preferably at about 1% w/v sodium chloride and having about 300 mOsm/L), physiologic saline solution (an aqueous solution comprising about 0.9 percent sodium chloride and about substantially isotonic with blood serum), Ringer's solution (an aqueous solution having about 130 mmol/L sodium, about 109 mmol/L chloride, about 28 mmol/L lactate, about 4 mmol/L potassium and about 1.5 mmol/L calcium), Shohl's solution (an aqueous solution comprising citric acid and sodium citrate), sodium hypochlorite solution (an aqueous solution having from about 3 to about 7 wt % sodium hypochlorite, preferably form about 4 to about 6 wt % sodium hypochlorite), or TAC solution (an aqueous solution comprising tetracaine, epinephrine and cocaine).

In a preferred embodiment of the present invention, the disinfecting agent dispersed, suspended or dissolved in a medical solution can be used as a wound irrigation system, a surgical irrigation system, a component of a wound dressing, a mouthwash, a gargle, a storage or preservative system, an injectable solution, an anti-itch solution, anti-bacterial solution, anti-fungal solution, anti- microbial solution, or antiseptic solution. Preferably, the medical solution comprises an aqueous system.

The disinfecting agent can be formulated into a cream, an ointment or salve, a liniment, or a gel. The disinfecting agent can be dispersed, suspended and/or dissolved in the cream, ointment, salve, paste, liniment, or gel formulation.

As used herein, a “cream” refers to an emulsion comprising an oil and water. The emulsion can be an oil in water emulsion or a water in oil emulsion. The oil to water ratio can be any ratio. Preferably, the portions of oil and water are substantially about equal. That is, the ratio of oil to water is about 1:1.

As used herein, an “ointment” or “salve” refers to a substantially viscous and/or semi-solid preparation. The ointment or salve can be formulated from a petroleum-based hydrocarbon (such as without limitation a paraffinic hydrocarbon), a natural-based hydrocarbon (such as without limitation a wool fat or beeswax), a vegetable oil (such as without limitation olive, coconut or arachis oils), or a man-made polymeric system (such as without limitation a polyether or macrogols). The ointment or salve can be in the form of an emulsion.

As used herein, a “liniment” refers to a less viscous form of an ointment, cream or gel. The term liniment can also refer to commonly used terms lotion or balm. The liniment can comprise one or more water, alcohol, acetone, or other quickly evaporating solvents.

As used herein, a “gel” refers to a thick solution. The solution can comprise an aqueous and/or alcoholic solution. Preferably, the gel comprises thick paste-like solution or semisolid emulsion.

A non-limiting example of disinfecting coating may comprise one or more rare earth-containing compositions; panthenol; and glycerin. More specifically, the one or more rare-containing compositions comprise from one of: preferably about 0.05 wt %, more preferably about 0.1 wt %, even more preferably about 0.2 wt %, even more preferably about 0.3 wt %, even more preferably about 0.4 wt %, even more preferably about 0.5 wt %, about 0.6 wt %, even more preferably about 0.7 wt %, even more preferably about 0.8 wt %, even more preferably about 0.9 wt %, even more preferably about 1 wt %, even more preferably about 2 wt %, even more preferably about 3 wt %, even more preferably about 4 wt %, even more preferably about 5 wt %, even more preferably about 6 wt %, even more preferably about 7 wt %, even more preferably about 8 wt %, even more preferably about 9 w%, even more preferably about 10 w%, even more preferably about 12 wt %, even more preferably about 14 wt %, even more preferably about 15 wt %, even more preferably about 16 wt %, even more preferably about 18 wt %, even more preferably about 20 wt %, or even more preferably about 25 wt % to one of: preferably about 0.1 wt %, more preferably about 0.2 wt %, even more preferably even more preferably about 0.3 wt %, even more preferably about 0.4 wt %, even more preferably about 0.5 wt %, even more preferably about 0.6 wt %, even more preferably about 0.7 wt %, even more preferably about 0.8 wt %, even more preferably about 0.9 wt %, even more preferably about 1 wt %, even more preferably about 2 wt %, even more preferably about 3 wt %, even more preferably about 4 wt %, even more preferably about 5 wt %, even more preferably about 6 wt %, even more preferably about 7 wt %, even more preferably about 8 wt %, even more preferably about 9 w%, even more preferably about 10 w%, even more preferably about 12 wt %, even more preferably about 14 wt %, even more preferably about 15 wt %, even more preferably about 16 wt %, even more preferably about 18 wt %, even more preferably about 20 wt %, even more preferably about 25 wt %, or even more preferably about 30 wt % of the disinfecting coating. In one preferred embodiment of the present invention, each of the one or more rare earth-containing compositions is at a concentration of from about 0.1 wt % to about 1.5 wt %.

More specifically, the pantheol comprises from one of preferably about 0 wt %, more preferably 0.01 wt %, even more preferably about 0.02 wt %, even more preferably about 0.03 wt %, even more preferably about 0.04 wt %, even more preferably about 0.05 wt %, even more preferably about 0.06 wt %, even more preferably about 0.07 wt %, even more preferably about 0.08 wt %, even more preferably about 0.09 wt %, even more preferably about 0.1 wt %, even more preferably about 0.2 wt %, even more preferably about 0.3 wt %, even more preferably about 0.4 wt %, even more preferably about 0.5 wt %, even more preferably about 0.6 wt %, even more preferably about 0.7 wt %, even more preferably about 0.8 wt %, even more preferably about 0.9 wt %, even more preferably about 1 wt %, even more preferably about 2 wt %, even more preferably about 3 wt %, even more preferably about 4 wt %, even more preferably about 5 wt %, even more preferably about 6 wt %, even more preferably about 7 wt %, even more preferably about 8 wt %, even more preferably about 9 w%, even more preferably about 10 w%, even more preferably about 12 wt %, even more preferably about 14 wt %, even more preferably about 15 wt %, even more preferably about 16 wt %, even more preferably about 18 wt %, even more preferably about 20 wt %, or about 25 wt % to one of: preferably about 0.01 wt %, more preferably about 0.02 wt %, even more preferably about 0.03 wt %, even more preferably about 0.04 wt %, even more preferably about 0.05 wt %, even more preferably about 0.06 wt %, even more preferably about 0.07 wt %, even more preferably about 0.08 wt %, even more preferably about 0.09 wt %, of about 0.1 wt %, even more preferably about 0.2 wt %, even more preferably about 0.3 wt %, even more preferably about 0.4 wt %, even more preferably about 0.5 wt %, even more preferably about 0.6 wt %, even more preferably about 0.7 wt %, even more preferably about 0.8 wt %, even more preferably about 0.9 wt %, even more preferably about 1 wt %, even more preferably about 2 wt %, even more preferably about 3 wt %, even more preferably about 4 wt %, even more preferably about 5 wt %, even more preferably about 6 wt %, even more preferably about 7 wt %, even more preferably about 8 wt %, even more preferably about 9 w%, even more preferably about 10 w%, even more preferably about 12 wt %, even more preferably about 14 wt %, even more preferably about 15 wt %, even more preferably about 16 wt %, even more preferably about 18 wt %, even more preferably about 20 wt %, even more preferably about 25 wt %, or about 30 wt % of the disinfecting coating. In a preferred embodiment of the present invention, the pantheol comprises from about 0.03wt % to about 5 wt % of the disinfecting coating.

More specifically, the glycerin comprises from: preferably about 0 wt %, more preferably 0.01 wt %, even more preferably about 0.02 wt %, even more preferably about 0.03 wt %, even more preferably about 0.04 wt %, even more preferably about 0.05 wt %, even more preferably about 0.06 wt %, even more preferably about 0.07 wt %, even more preferably about 0.08 wt %, even more preferably about 0.09 wt %, even more preferably about 0.1 wt %, even more preferably about 0.2 wt %, even more preferably about 0.3 wt %, even more preferably about 0.4 wt %, even more preferably about 0.5 wt %, even more preferably about 0.6 wt %, even more preferably about 0.7 wt %, even more preferably about 0.8 wt %, even more preferably about 0.9 wt %, even more preferably about 1 wt %, even more preferably about 2 wt %, even more preferably about 3 wt %, even more preferably about 4 wt %, even more preferably about 5 wt %, even more preferably about 6 wt %, even more preferably about 7 wt %, even more preferably about 8 wt %, even more preferably about 9 w%, even more preferably about 10 w%, even more preferably about 12 wt %, even more preferably about 14 wt %, even more preferably about 15 wt %, even more preferably about 16 wt %, even more preferably about 18 wt %, even more preferably about 20 wt %, or even more preferably about 25 wt % to one of: preferably about 0.01 wt %, more preferably about 0.02 wt %, even more preferably about 0.03 wt %, even more preferably about 0.04 wt %, even more preferably about 0.05 wt %, even more preferably about 0.06 wt %, even more preferably about 0.07 wt %, even more preferably about 0.08 wt %, even more preferably about 0.09 wt %, even more preferably about 0.1 wt %, even more preferably about 0.2 wt %, even more preferably about 0.3 wt %, even more preferably about 0.4 wt %, even more preferably about 0.5 wt %, even more preferably about 0.6 wt %, even more preferably about 0.7 wt %, even more preferably about 0.8 wt %, even more preferably about 0.9 wt %, even more preferably about 1 wt %, even more preferably about 2 wt %, even more preferably about 3 wt %, even more preferably about 4 wt %, even more preferably about 5 wt %, even more preferably about 6 wt %, even more preferably about 7 wt %, even more preferably about 8 wt %, even more preferably about 9 w%, even more preferably about 10 w%, even more preferably about 12 wt %, even more preferably about 14 wt %, even more preferably about 15 wt %, even more preferably about 16 wt %, even more preferably about 18 wt %, even more preferably about 20 wt %, even more preferably about 25 wt %, or even more preferably about 30 wt % of the disinfecting coating. In a preferred embodiment of the present invention, the pantheol comprises from about 0 wt % to about 5 wt % of the disinfecting coating. In another preferred embodiment of the present invention, the glycerin comprises from about 0 wt % to about 5 wt % of the coating.

Any of the therapeutic formulations of the present invention may be applied topically to the skin or to the various mucous membranes of an animal, including but not limited to those of the oral, nasal, vaginal or rectal cavities, to prevent the effects of exogenous irritants upon these surfaces. The therapeutic formulations of the invention may be used as disinfectants, for example handscrubs to be used prior to donning surgical gloves.

Any of the therapeutic formulations of the present invention may be applied as coatings to articles, for example barrier articles, and as such may, in an article having more than one surface, coat at least one surface (the entire surface or a portion thereof) of the article. More specifically, as an embodiment, a coating according to the invention may be applied to one or both of an inner and outer surfaces of a glove or any other article cover at a portion of the body. Different coatings may be applied to each surface. A coating may be applied over a portion of a surface, for example, but not by way of limitation, on the inner surface of one or more fingertip of a glove.

Various therapeutic formulations of the present invention may comprise an emollient, such as, but not limited to, PEG 20 almond glycerides, Probutyl DB-10, Glucam P-20, Glucam E-10, Glucam P-10, Glucam E-20, Glucam P-20 distearate, Procetyl-10 (Croda), Incroquat, glycerin, propylene glycol, cetyl acetate, and acetylated lanolin alcohol, cetyl ether, myristyril ether, hydroxylated milk glycerides, polyquaternium compounds, copolymers of dimethyl dialyl ammonium chloride and acrylic acid, dipropylene glycol methyl ethers, polypropylene glycol ethers and silicon polymers. Other suitable emollients may include hydrocarbon-based emollients such as petrolatum or mineral oil, fatty ester-based emollients, such as methyl, isopropyl and butyl esters of fatty acids such as isopropyl palmitate, isopropyl myristate, isopropyl isostearate, isostearyl isostearate, diisopropyl sebacate, and propylene dipelargonate, 2-ethylhexyl isononoate, 2-ethylhexyl stearate, C₂-C₁₆ fatty alcohol lactates such as cetyl lactate and lauryl lactate, isopropyl lanolate, 2-ethylhexyl salicylate, cetyl myristate, oleyl myristate, oleyl stearate, oleyl oleate, hexyl laurate, and isohexyl laurate. Further emollients include lanolin, olive oil, cocoa butter, and shea butter. The present invention provides for the incorporation, into formulations and coatings, of one or more emollient solvent. Preferred emollient solvents of the invention include octoxyglycerin (Sensiva.RTM.), pentylene glycol, 1,2 hexanediol and caprylyl glycol, for example, and not by way of limitation, at a concentration of up to 5 percent or up to 3 percent.

Various embodiments of the therapeutic formulation may comprise a stabilizing agent and/or an antioxidant (which may be at a concentration of 0.2-1%), such as but not limited to vitamin C (ascorbic acid) or vitamin E (tocopherol).

Various embodiments the therapeutic formulation of the present invention may comprise a thickening agent, such as but not limited to the following (at a preferred concentration of 0.6-2%): stearyl alcohol, cationic hydroxy ethyl cellulose (U Care JR30; Amerchol), hydroxy propyl methyl cellulose, hydroxy propyl cellulose (Klucel), Polyox N-60K, chitosan pyrrolidone carboxylate (Kytamer), behenyl alcohol, zinc stearate, Crodamol STS (Croda) or an emulsifying wax, such as but not limited to, Incroquat and Polawax. Other thickening and/or gelling agents suitable for incorporation into the formulations or ointments described herein include, for example, an addition polymer of acrylic acid, a resin such as Carbopol™ 2020, guar gum, acacia, acrylates/steareth-20 methacrylate copolymer, agar, algin, alginic acid, ammonium acrylate co-polymers, ammonium alginate, ammonium chloride, ammonium sulfate, amylopectin, attapulgite, bentonite, C₉-C₁₅ is alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, crodomol, crothix, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C₂₉-C₇₀ acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C₆-C₁₄ olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof.

An embodiment of the therapeutic formulation may comprise phenoxyethanol (0.3-1.0%) as a solubilizing agent.

An embodiment of therapeutic formulation of the present invention may comprise a humectant, such as but not limited to glycerin, panthenol, Glucam P20, 1-2-propylene glycol, dipropylene glycol, polyethylene glycol, 1,3-butylene glycol, or 1,2,6-hexanetriol.

Another embodiment of the therapeutic formulation of the present invention may comprise one or more preservative agent, preferably at a total concentration between 0.05 wt % and 5 wt % or between 0.05 wt % and 2 wt % or between 0.1 wt % and 2 wt %. Examples of preferred preservative agents include, but are not limited to, chlorhexidine gluconate (CHG), benzalkonium chloride (BZK), or iodopropynylbutyl carbamate (IPBC; Germall plus). Further examples of preservative agents include, but are not limited to, iodophors, iodine, benzoic acid, dihydroacetic acid, propionic acid, sorbic acid, methyl paraben, ethyl paraben, propyl paraben, butyl paraben, cetrimide, quaternary ammonium compounds, including but not limited to benzethonium chloride (BZT), dequalinium chloride, biguanides such as chlorhexidine (including free base and salts (see below)), PHMB (polyhexamethylene biguanide), chloroeresol, chlorxylenol, benzyl alcohol, bronopol, chlorbutanol, ethanol, phenoxyethanol, phenylethyl alcohol, 2,4-dichlorobenzyl alcohol, thiomersal, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, and pharmaceutically acceptable salts thereof.

Pharmaceutically acceptable chlorhexidine salts of the present invention that may be used as preservative agents according to the invention include, but are not limited to, chlorhexidine palmitate, chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-.alpha.-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynapthoate, and chlorhexidine embonate. Chlorhexidine free base is a further example of a preservative agent.

These and further examples of preservation agents useful in this invention can be found in such references as GOODMAN AND GILMAN′S THE PHARMACOLOGICAL BASIS OF THERAPEUTICS (Alfred Goodman Gilman, Theodore W. Rall, Alan S. Nies, Palmer Taylor, eds., Pergamon Press 1990) (1941), the contents of which are hereby incorporated by reference.

An embodiment of the therapeutic formulation of the present invention may comprise a neutralizing agent to neutralize carboxyl groups present in one or more other component, such as carboxyl groups in a thickening agent. Suitable neutralizing agents include diisopropylamine and triethanolamine.

Various embodiments of the therapeutic formulation of the present invention may comprise a surfactant. The surfactant may be an anionic surfactant, a cationic surfactant, an ampholytic surfactant, or a nonionic surfactant. Examples of nonionic surfactants include polyethoxylates, fatty alcohols (e.g., ceteth-20 (a cetyl ether of polyethylene oxide having an average of about 20 ethylene oxide units) and other “BRIJ″™ nonionic surfactants available from ICI Americas, Inc. (Wilmington, Del.)), cocamidopropyl betaine, alkyl phenols, fatty acid esters of sorbitol, sorbitan, or polyoxyethylene sorbitan. Suitable anionic surfactants include ammonium lauryl sulfate and lauryl ether sulfosuccinate. Preferred surfactants include lauroyl ethylenediamine triacetic acid sodium salt at a concentration between about 0.5-2.0%, Pluronic F87 at about 2.0%, Masil SF-19 (BASF) ans incromide. Suitable concentrations of surfactant are between about 0.05% and 2%.

Water used in the therapeutic formulation embodiments of the present invention is preferably deionized water having a neutral pH. When used in hydroalcoholic gel compositions, the concentration of water should be suitable to dissolve the hydrogels according to the invention. Alcohols that may be used according to the invention include but are not limited to ethanol and isopropyl alcohol.

Various embodiments of the therapeutic formulations of the present invention may comprise additional additives, including but not limited to a silicone fluid (such as dimethicone or cyclomethicone), a silicone emulsion, dyes, fragrances, pH adjusters, including basic pH adjusters such as ammonia, mono-, di- and tri-alkyl amines, mono-, di- and tri-alkanolamines, alkali metal and alkaline earth metal hydroxides (e.g., ammonia, sodium hydroxide, potassium hydroxide, lithium hydroxide, monoethanolamine, triethylamine, isopropylamine, diethanolamine and triethanolamine); acid pH adjusters such as mineral acids and polycarboxylic acids (e.g., hydrochloric acid, nitric acid, phosphoric acid, sulfuric acid, citric acid, glycolic acid, and lactic acid); vitamins such as vitamin A, vitamin E and vitamin C; polyamino acids and salts, such as ethylenediamine tetraacidic acid (EDTA), preservatives such as Germall plus and DMDM hydantoin, and sunscreens such as aminobenzoic acid, arobenzone, cinoxate, diioxybenzone, homosalate, menthyl anthranilate, octocrylene, octyl methoxycinnamate, octyl salicylate, oxybenzoate, padimate O, phenylbenzimidazole, sulfonic acid, sulisobenzone, titanium dioxide, trolamine salicylate and zinc oxide.

Various embodiments of the therapeutic formulations of the present invention may comprise an essential oil (“EO”), which is a volatile oil obtained from a plant or an animal source that comprises one or more active agent (also referred to herein as an Isolated Component or “IC”) which may be, for example but not by way of limitation, a monoterpene or sesquiterpene hydrocarbon, alcohol, ester, ether, aldehyde, ketone, or oxide. Examples of these E0s include, but are not limited to, almond oil, ylang-ylang oil, neroli oil, sandalwood oil, frankincense oil, peppermint oil, lavender oil, jasmine absolute, geranium oil bourbon, spearmint oil, clove oil, lemongrass oil, cedarwood oil, balsam oils, and tangerine oil. Alternatively, the present invention provides for the use of active agents found in essential oils (ICs) such as, but not limited to, 1-citronellol, .alpha.-amylcinnamaldehyde, lyral, geraniol, farnesol, hydroxycitronellal, isoeugenol, eugenol, eucalypus oil and eucalyptol, lemon oil, linalool, and citral. The concentrations of EO or IC may be between about 0.3 wt % and 1 wt % or between about 0.1 wt % and 0.5 wt % or between 0.5 wt % and 2 wt %.

A hydrogel, as used in any of the therapeutic formulation embodiments of the present invention may comprise hydroxypropylmethyl cellulose, cationic hydroxyethyl cellulose (U-care polymers), ethyl cellulose, hydroxypropyl cellulose, hydroxymethyl cellulose, hydroxypropyl methyl cellulose (methocell K4MS) carboxy methyl cellulose, polyethylene oxide (polyox resins), or chitosan pyrrolidone carboxylate (Kytomer PC). In addition, it has been discovered that alcohol used to form the hydroalcoholic gel is not trapped in the hydroalcoholic gel composition and is therefore available for rapid and long-term action. The hydrogel may be present in a concentration between 0.1-1.0%, and preferably is a cationic hydroxyethyl cellulose (U-care polymers) in a concentration between 0.05-0.5%, most preferably 0.2%.

Alcohols that may be used in any of the hydroalcoholic gel embodiments of the present invention include aliphatic alcohols, including, but not limited to, ethanol, isopropyl alcohol, n-propyl alcohol, and mixtures thereof; fatty alcohols, including, but not limited to, cetyl alcohol, myristol alcohol, stearyl alcohol, octyl alcohol, decyl alcohol and lauryl alcohol, and mixtures thereof; and hexanol. The concentration of alcohol may be between 30% and 95%, preferably between 40% and 70%; preferably the aliphatic alcohols is ethanol or isopropyl alcohol at a concentration between and 60% and 95%. When present, the concentration of fatty alcohols is preferably between 0.5% and 5.0%; and, when present, the concentration of hexanol is preferably between 3% and 10%, more preferably 5%. These same emulsifiers may be used in other formulations of the invention as well.

Hydroalcoholic gel embodiments of the present invention may optionally comprise an emollient and/or humectant such as the emollients and humectants discussed above, preferably one or more of PEG 20 Almond Glycerides, Probutyl DB-10, Glucam P20, Glucam E-10, Glucam P-10, Glucam E-20, Glucam P-20 distearate, glycerin, propylene glycol, octoxyglycerin (Sensiva™), cetyl acetate and acetylated lanolin alcohol (Acetulan), cetyl ether (PPG-10), myristyl ether (PPG-3), hydroxylated milk glycerides (Cremerol HMG), polyquaternium compounds (U-care compounds), chitosan (Kytamer), copolymer of dimethyl dialyl ammonium chloride and acrylic acid (Merquat), dipropylene glycol methyl ethers (Dowanol DPM Dow Corning), or polypropylene glycol ethers (Ucon 50-HB-660, Union Carbide). Preferably the emollient is present at a concentration of 3% or less, such that the viscosity of the composition is preferably less than 2000 centipoise at 20-40 degrees Celsius, more preferably between 0.2 and 3%.

Hydroalcoholic gel embodiments of the present invention may optionally comprise a surfactant and/or emulsifier, such as the emulsifiers and surfactants discussed above, and preferably a non-ionic or cationic self-emulsifying wax that is soluble in alcohol at ambient temperature. Suitable surfactant/emulsifiers include but are not limited to Incroquat Behenyl TMS, Incroquat Behenyl TMS-50, Polawax, stearyl alcohol and cetearyl alcohol. These emulsifiers may be present at a concentration between 0.05-3.0%. Preferred emulsifiers include Incroquat Behenyl TMS, which is a mild cationic emulsifier as well as an excellent conditioner, and Polawax, which is a non-ionic self emulsifying wax, individually at a concentration of between 0.05-0.5%, and in combination at a concentration of between 0.05-0.5%, more preferably in combination at a concentration ratio of approximately 1:1. If more than one emulsifier is used, it is preferred that the total concentration of emulsifiers present is between 0.05-0.5%.

Any hydroalcoholic gel therapeutic formulation embodiment may optionally comprise a silicone polymer such as, but not limited to, one or more than one polydimethylsiloxane polymer (Dow Corning 225 Silicone Fluid), dimethiconol fluid in dimethicone (Dow Corning 1403 Silicone Fluid), cyclomethicone and dimethicone copolyl (Dow Corning 3225C Silicone Fluid), or silicone glycol (BASF 1066 DCG polyol). Preferred concentrations of silicone polymer are between about 0.1-1.0%.

Any of the hydroalcoholic gel embodiments of the present invention may optionally comprise an emollient solvent such as, but are not limited to, those listed above or one or more than one glycidyl ethers having alkyl chains up to and including 18 carbon molecules and ethoxylates and propoxylates thereof, glyceryl ethers having alkyl chains up to and including 18 carbon molecules and ethoxylates and propoxylates thereof, mono- and diglyceryl ethers having alkyl chains up to and including 18 carbon molecules and ethoxylates and propoxylates thereof, ethoxylate and propoxylate ethers, ethoxy diglycol esters, ethyl hexyl alcohol propoxylate, propylene glycol esther ethoxylates or propoxylates, or, preferably Arlamol (Altas). Preferred concentrations of emollient solvent are between 0.5-5%.

Any of the hydroalcoholic gel formulations of the present invention may optionally comprise a thickening agent, such as, but not limited to, a thickening and/or gelling agent discussed above, preferably behenyl alcohol, crodomol, or crothix. Preferred concentrations of thickening agent are between 0.05-10%. Gelling agents such as Caropol are not preferred due to their high viscosity and their requiring neutralizing agents to neutralize the gelling agent with alkaline materials.

In non-limiting embodiments, any composition of the present invention may comprise a pre-existing formulation, such as a commercially available cream, liquid, gel or lotion. Examples of commercially available formulations that may be so used include, but are not limited to, personal lubricants sold under the trade names KY JELLY™, ASTROGLIDE™, and PREVACARE™ and lotions sold under the trade names SOFT-SENSE™, LOTION SOFT™, CUREL™, and KERI™ SOFT-SENSE (Johnson & Son, Inc., Racine, Wis.) is known to contain purified water, glycerin USP, distearyldimonium chloride, petrolatum USP, isopropyl palmitate, 1-hexadecanol, tocopheryl acetate (vitamin E USP), dimethicone, titanium dioxide USP, methyl paraben, propyl paraben, sodium chloride, and fragrance. LOTION SOFT™ (Calgon Vestal, St. Louis, Mo.) is a nonionic moisturizing lotion which is known to contain mucopolysaccharide. CUREL™ (Bausch & Lomb Incorporated, Rochester, N.Y.) is known to contain deionized water, glycerin, quaternium-5, petrolatum, isopropyl palmitate, 1-hexadecanol, dimethicone, sodium chloride, fragrance, methyl paraben, and propyl paraben.

A non-limiting example of therapeutic formulation is a hydrogel composition comprising a disinfecting agent and a hydrogel. The disinfecting agent comprises one or more rare-earth containing compositions. Preferably, the disinfecting agent can comprise up to about 0.5 wt %, up to about 1 wt %, up to about 2wt %, up to about 3 wt %, up to about 4 wt %, up to about 5wt %, up to about 6 wt %, up to about 7 wt %, up to about 8 wt %, up to about 9 wt %, up to about 10 wt %, up to about 12 wt %, up to about 14 wt %, up to about 15 wt %, up to about 16 wt %, up to about 18 wt %, up to about 20 wt %, up to about 30 wt %, up to about 35 wt %, up to about 40 wt %, up to about 45 wt %, up to about 50 wt % of the hydrogel composition.

The hydrogel composition can optionally contain humectants (e.g. glycerin) and may or may not contain a polymer of an acid (e.g., polyacrylic acid, or an acid forming compound such as an anhydride).

The hydrogel may be reversible or irreversible hydrogel. The components of a reversible hydrogel dissolve in water. The components of an irreversible hydrogel gel do not dissolve in water due to the presence of cross-linking agents (i.e. cross-linkers) which provide, depending on the amount used, a certain amount of irreversible links.

Cross-linkers enhance the ability of the hydrogel compositions to maintain their original shape. Examples of cross-linkers which are suitable for use in the composition include glutaraldehyde, genipin, aziridine derivatives, carbodimid derivatives, colloidal silica, colloidal alumina, colloidal titanium dioxide, polyaminosilanes, epoxies, primary polyamines, dialdehydes, polyaldehydes from acrolein reaction products, paraformaldehyde, acrylamides, polyethylenimines, and combinations thereof. Cross-linkers can be used in any amount which provides the hydrogel with desired consistencies. For example, the hydrogel and/or the hydrogel composition can comprise up to about 2 wt %, up to about 3 wt %, up to about 4 wt %, up to about 5 wt %, or up to about 8 wt % of a cross-linker.

The hydrogel comprising the hydrogel composition may comprise a poly(N-vinyl lactam), a polysaccharide, and water. Preferably, the range of the ratio of the amount by weight of the poly(N-vinyl) lactam to the amount by weight of the polysaccharide may have an upper boundary of approximately 75:1. Examples of other upper boundaries include about 1; 50:1; 30:1; 20:1; 15:1; 13:1; 12:1; and 1:2. Preferably, the range of the ratio of the amount by weight of the poly(N-vinyl) lactam to the amount by weight of the polysaccharide may have a lower boundary of approximately 1:10. Examples of other lower boundaries may include about 1:5; 1:3, 1:1; 5:1; 12:1; 13:1; 15:1; 20:1; 30:1; and 50:1.

The poly(N-vinyl lactam) of the hydrogel may be any type of poly(N-vinyl lactam), such as, for example, a homopolymer, a copolymer, or a terpolymer of N-vinyl lactam, or mixtures thereof. Examples of poly(N-vinyl lactam) polymers suitable for use in the hydrogel composition include N-vinylpyrrolidone, N-vinylbutyrolactam, N-vinylcaprolactam, and mixtures thereof. An example of a preferred poly(N-vinyl lactam) homopolymer is polyvinylpyrrolidone (PVP). Examples of poly(N-vinyl lactam) copolymers and terpolymers include N-vinyl lactam polymers which are copolymerized with vinyl monomers. Examples of vinyl monomers include acrylates, hydroxyalkylacrylates, methacrylate, acrylic acids, methacrylic acids, acrylamides, and mixtures thereof. The copolymerization of the N-vinyl lactams with vinyl monomers allows for modification of the consistency of the hydrogel compositions.

The polysaccharide may be any polysaccharide and/or any polysaccharide derivative. Examples of a suitable polysaccharide include chitin; deacetylated chitin; chitosan; chitosan salts; chitosan sorbate; chitosan propionate; chitosan lactate; chitosan salicylate; chitosan pyrrolidone carboxylate; chitosan itaconate; chitosan niacinate; chitosan formate; chitosan acetate; chitosan gallate; chitosan glutamate; chitosan maleate; chitosan aspartate; chitosan glycolate; quaternary amine substituted chitosan salts; N-carboxymethyl chitosan; O-carboxymethyl chitosan; N,—O-carboxymethyl chitosan; equivalent butyl chitosan derivatives; cellulosics, alkylcellulose; nitrocellulose; hydroxypropylcellulose; starch; starch derivatives; methyl gluceth derivatives; collagen, alginate; hialuronic acid; heparin; heparin derivatives; and combinations thereof.

The combined poly(N-vinyl lactam) and polysaccharide is hydrophilic, and is capable of absorbing many times its weight in water. The water content of the hydrogel can vary depending on the particular use of the hydrogel composition, as would be known by a skilled artisan. Preferably, the range of the water content in either the hydrogel or hydrogel compositions have an upper boundary of about 90 wt % water. Examples of other upper boundaries include about 75 wt % water and 65 wt % water. Preferably, the range of the water content in either the hydrogel or the hydrogel composition has a lower boundary of about 25 wt %. Examples of other lower boundaries include about 45 wt % water and 55 wt %. As the water content increases, the hydrogel and/or hydrogel composition become softer. Optionally, an alcohol may replace at least some of the water comprising the hydrogel and/or hydrogel composition. Approximately 15 wt % to 75 wt %, 35 wt % to 65 wt %, or 45 wt % to 55 wt % of the water can be replaced with alcohol. Preferred examples of alcohols include ethyl alcohol and isopropyl alcohol.

Optionally, the hydrogel composition can further comprise at least one consistency modifying agent, a performance modifying agent, a cross-linker, or mixtures thereof. Up to approximately 5 wt %, 10 wt %, 20 wt %, 30 wt %, 40 wt %, 50 wt %, 60 wt %, 70 wt %, 80 wt %, or 90 wt % of the poly(N-vinyl lactam) can be replaced with the consistency and/or performance modifying agents. For example, in a formulation comprising polyvinyl pyrrolidone (PVP) and chitosan, or chitosan derivatives, preferably about 50 wt % of the PVP is replaced with consistency and/or performance modifying agents. Examples of preferred consistency modifying and/or performance modifying agents include polyvinyl alcohol; polyvinyl acetate; polyethylenoxide, poly(2-hydroxyethyl methacrylate); methyl vinyl ether-co-maleic anhydride; poly(ethylene-co-vinyl acetate); polyethylene glycol diacrylate; poly(N-isopropyl acrylamide); polyurethane; dimethicone; polyglycol ester copolymers, adhesive prepolymers, polyethylenimine; polypeptides; keratins; copolymers of polyvinylpyrrolidone/polyethyleneimine; polyvinylpyrrolidone/polycarbamyl/-polyglycol ester (Aquamere™ H-1212, H-1511, H-2012, A-1212); polyvinylpyrrolidone/dimethylaminoethylmethacrylate/polycarbamyl/polyglycol ester (Aquamere™ C-1011, C-1031); polyvinyl-pyrrolidone/dimethiconylacrylate/polycarbamyl/-polyglycol ester (Aquamere™ S2011, S-2012); (PECOGEL™ equivalents of the Aquamere™ products); lecithin; and copolymers, derivatives and combinations thereof.

### Cleaning Composition and Method for Making the Same

Yet another embodiment of the present invention is a cleaning composition comprising the disinfecting agent. The cleaning agent can comprise a fluid or solid. The cleaning composition comprises the disinfecting agent and one or more of a surfactant and/or wetting agent. The one or more surfactants can comprise any surfactant. Preferably, the surfactant comprises one of anionic surfactant, a cationic surfactant or a non-ionic surfactant. The cleaning composition can further comprise builders, binders, and fillers. The disinfecting agent can be dispersed, suspended or dissolved in the cleaning agent. The cleaning agent can be a bar soap, a liquid soap, a soap concentrate, a detergent (such as, but not limited to a laundry, household, industrial, dish, or sterile processing detergent), a surgical prepare, a personal care product (such as but not limited to face, hair, person, beauty, acne, or foot care cleaning product), or a home or farm care product (such as, but not limited to a hard surface cleaner, a floor care product, a carpet care product, an air care product, a bathroom care product, a nursery care product, an upholstery car product, a pet care product, a veterinary product, an agricultural care product (such as, but not limited to the cleaning of farm animals, farm product, agriculture structures or equipment).

Any process known within the art can be used to make the cleaning composition. Soluble forms of the disinfecting agent can be added to the cleaning composition in a dry and/or in dissolved form. Insoluble forms of the disinfecting agent can be suspended and/or dispersed in the cleaning composition.

A non-limiting example of a hard surface cleaning composition may comprise an aqueous liquid cleaning composition that includes:

(a) a disinfecting agent comprising one or more rare earth containing compositions;

(b) a water-soluble or water-dispersible copolymer having: (i) a first monomer that is capable of forming a cationic charge on protonation selected from the group consisting of an N-alkyl acrylamide, N-alkyl(alkyl)acrylamide, N-aryl acrylamide, N-aryl(alkyl)acrylamide, N-alkyl(aryl)acrylamide, N,N-di-alkyl acrylamide, N,N-di-alkyl(alkyl)acrylamide, N,N-di-alkyl(aryl)acrylamide, N,N-di-aryl acrylamide, N,N-di-aryl(alkyl)acrylamide, N,N-di-aryl(aryl)acrylamide, N-alkylamino alkyl acrylamide, N-alkylamino alkyl(alkyl)acrylamide, N-alkylamino alkyl(aryl)acrylamide, N-arylamino alkyl acrylamide, N-arylamino alkyl(alkyl)acrylamide, N-arylamino alkyl(aryl)acrylamide, N,N-di-alkylamino alkyl acrylamide, N,N-di-alkylamino alkyl(alkyl)acrylamide, N,N-di-alkylamino alkyl(aryl)acrylamide, N,N-di-arylamino alkyl acrylamide, N,N-di-arylamino alkyl(alkyl)acrylamide, N,N-di-arylamino alkyl(aryl)acrylamide, and combinations thereof, wherein said alkyl moiety is a radical independently selected from the group consisting of a C₁ to C₆ saturated alkyl, vinyl, C₃ to C₆ unsaturated alkylene radical, and combinations thereof, wherein said aryl moiety is a radical independently selected from the group consisting of a benzyl, phenyl, styryl, hydroxyphenyl, alkylbenzyl, alkylphenyl radical, and combinations thereof; (ii) second monomer that is acidic and that is capable of forming an anionic charge in the compositions; (iii) optionally, a third monomer that has an uncharged hydrophilic group; and (iv) optionally, a fourth monomer that is hydrophobic;

(c) optionally, an organic solvent;

(d) a surfactant; and

(e) optionally, an adjuvant;

wherein said copolymer is capable of forming an invisible film on a treated surface exhibiting a water contact angle of less than 10 degrees and a thickness of less than about 100 nm on said treated surface after a cleaning operation.

More over, the example includes a method of disinfecting a hard surface and depositing an invisible protective copolymer film that comprises the steps of:

(a) applying a cleaning composition comprising a disinfecting agent comprising one or more rare earth-containing compositions, a water-soluble or water dispersible copolymer onto the hard surface;

(b) removing the cleaning composition whereby a layer of the disinfecting agent remains on the hard surface; and

(c) allowing the layer to dry to thereby leave a copolymer film on the hard surface which contains the disinfecting agent and at least some of the copolymer. The co-polymer can have a first monomer that is capable of forming a cationic charge on protonation selected from the group consisting of an N-alkyl acrylamide, N-alkyl(alkyl)acrylamide, N-aryl acrylamide, N-aryl(alkyl)acrylamide, N-alkyl(aryl)acrylamide, N,N-di-alkyl acrylamide, N,N-di-alkyl(alkyl)acrylamide, N,N-di-alkyl(aryl)acrylamide, N,N-di-aryl acrylamide, N,N-di-aryl(alkyl)acrylamide, N,N-di-aryl(aryl)acrylamide, N-alkylamino alkyl acrylamide, N-alkylamino alkyl(alkyl)acrylamide, N-alkylamino alkyl(aryl)acrylamide, N-arylamino alkyl acrylamide, N-arylamino alkyl(alkyl)acrylamide, N-arylamino alkyl(aryl)acrylamide, N,N-di-alkylamino alkyl acrylamide, N,N-di-alkylamino alkyl(alkyl)acrylamide, N,N-di-alkylamino alkyl(aryl)acrylamide, N,N-di-arylamino alkyl acrylamide, N,N-di-arylamino alkyl(alkyl)acrylamide, N,N-di-arylamino alkyl(aryl)acrylamide, and combinations thereof.

The alkyl moiety can comprise a radical independently selected from the group consisting of a C₁ i to C₆ saturated alkyl, vinyl, C₃ to C₆ unsaturated alkylene radical, and combinations thereof; (ii) a second monomer that is acidic and that is capable of forming an anionic charge in the compositions; (iii) optionally, a third monomer that has an uncharged hydrophilic group; and (iv) optionally, a fourth monomer that is hydrophobic. The aryl moiety can comprise a radical independently selected from the group consisting of a benzyl, phenyl, styryl, hydroxyphenyl, alkylbenzyl, alkylphenyl radical, and combinations thereof. Preferably, the copolymer film exhibits a water contact angle of less than 10 degrees. Furthermore, the copolymer film can have a thickness of less than about 100 nm on the hard surface

### A Cellulosic-Containing Product and a Method for Making the Same

Yet other embodiment of the present invention is a cellulosic-containing product containing the disinfecting agent. Non-limiting examples of the cellulosic-containing product include a wipe, a filter, a food packing system, a tissue, a sheet of paper, a paper towel, a sheet of paperboard, a label, a sheet decor paper, an adhesive paper, a paper mask, a paper gown, a paper cap, a sheet of toilet paper, a paper toilet seat cover, a roll of wallpaper, a sheet of wallboard, a roll or sheet of cardboard, a wood product, a composite wood product, a particle board, a wood plastic composite, an acoustical panel, a wood filled plastic, or a wood flour.

The disinfecting agent can be incorporated in a paper product before, during or after the formation of wet paper matte. The disinfecting agent, in the form of soluble and/or insoluble compositions, can be added to the paper pulp prior to formation of the paper matte. The insoluble form of the disinfecting agent can be retained within the formed paper matte to form a paper product comprising the disinfecting agent. The soluble form of the disinfecting can be retained within one or both of the cellulosic fibers comprising the paper pulp and/or within the water retained by the wet-casted paper web. When the water is removed from wet-casted paper web is dried, the soluble remains behind and is retained within the dried paper product.

### A Polymeric Material and a Method for Making the Same

Yet another embodiment of the present invention is a polymeric material comprising the disinfecting agent. The polymeric material can be any polymeric material having the disinfecting agent contained within the continuous phase of the polymeric material and/or as a coating contained on one or more surfaces of the polymeric material. The disinfecting agent may be uniformly or non-uniformly distributed throughout the polymeric material. For example, the disinfecting material may be distributed more density on one or more surfaces of the polymeric material. The polymeric phase of the synthetic or natural polymeric materials identified above including homo-polymers, co-polymers, block-polymers, mixtures, combinations and polymeric alloys thereof.

Non-limiting examples of polymeric material comprising the disinfecting agent are: syringe barrels and/or plungers; plastic food wrap; plastic sterile wraps; plastic wound bandage pad; plastic wound bandage covers; medical tubing; polymeric fibers, threads and yarns; and any polymeric material used as a structural component requiring antimicrobial properties.

The disinfecting agent can be incorporated in the polymeric material before, during or after the formation the polymeric material. The disinfecting agent, in the form of soluble and/or insoluble rare earth-containing compositions, can be incorporated into the polymeric material during the polymerization process. Polymerization process refers to a homo-polymerization process, a co-polymerization process, a cross-linking process, or any combinations thereof.

In one embodiment, the disinfecting agent, in the form of soluble and/or insoluble rare earth-containing compositions, can be incorporated into the polymeric material during an extrusion process, casting process, a blending process, a molding process, a blow molding process, a reactive injection molding process, a laminating process, or any combination thereof. Preferably, the disinfecting agent is incorporated into the polymeric material under one or more of shear, high temperature, and high pressure. Furthermore, the polymer material is preferably in one of a thermoplastic and/or liquid state during the incorporation of disinfecting agent into the polymeric material.

A non-limiting example of a method for incorporating one or more rare earth-compositions into a polymeric composition comprises a continuous hydrophobic phase comprising a mixture comprising: a hydrophobic liquid comprising mineral oil; and a hydrophobic thermoplastic elastomeric polymer; absorbent hydrophilic microparticles dispersed within the hydrophobic liquid; and a disinfecting agent.

More specifically, the polymer composition comprises a mixture comprising: mineral oil; and a hydrophobic thermoplastic elastomeric polymer selected from the group consisting of styrene-isoprene-styrene (SIS), styrene-butadiene-styrene (SBS), styrene-ethylene-propylene-styrene (SEPS), styrene-ethylene-butylene-styrene (SEBS), and combinations thereof; absorbent hydrophilic microparticles dispersed within the mineral oil, the hydrophilic microparticles comprise a crosslinked carboxylic acid-containing organic polymer; and a disinfecting agent dispersed in the hydrophilic microparticles.

The disinfecting agent comprises one or more rare earth-containing compositions. Preferably, the disinfecting agent comprises particles having an average particle size of less than about one micron.

Preferably, the hydrophilic microparticles comprise a cross-linked carboxylic acid-containing organic polymer.

The polymer composition can be nonadherent or adherent. Preferably, the polymer composition contains no more than about 1 wt % water based on the total weight of the composition.

The hydrophilic polymer can be an amine-containing polymer such as, without limitation: poly(quaternary amines), polylactams, polyamides, and combinations thereof. Preferably, the polymer composition optionally includes a second organic polymer, thereby forming a mixture or blend of polymers. The second organic polymer is preferably a hydrophobic material. In one embodiment, the hydrophobic material forms a continuous matrix and the hydrophilic polymer forms a discontinuous phase (e.g., microparticles). The hydrophobic material can, preferably, form a discontinuous phase and the hydrophilic polymer forms a continuous matrix, a bi-continuous, or co-continuous phase with the hydrophilic polymer. The hydrophilic polymer can comprise particles, in the form of microparticles or a dispersion, such as a continuous hydrophobic liquid phase (e.g., mineral oil) and hydrophilic polymer particles dispersed within the hydrophobic liquid phase. Suitable examples of the hydrophobic polymer include, without limitation, SALCARE SC95 and SC96 which include a cationic homopolymer of the methyl chloride quaternary salt of 2-(dimethylamino)ethyl methacrylate. Other suitable examples include SALCARE SC91, a copolymer of sodium acrylate and acrylic acid. The hydrophilic polymers can be used in a variety of combinations. The total amount of hydrophilic polymer(s) is preferably at least 1 wt-%, and more preferably, at least 5 wt %, based on the total weight of the polymer composition. The total amount of hydrophilic polymer(s) (e.g., microparticles) is preferably at most 60 wt %, based on the total weight of the polymer composition.

The disinfecting agent can be present in the polymer composition in an amount to produce a desired effect. A preferred weight ratio of the disinfecting agent to hydrophilic polymers is at least 0.1 wt % based on the total weight of the hydrophilic polymer. Although there is essentially no upper limit, a preferred weight ratio is no more than 10 wt %.

The polymer compositions can include one or more secondary organic polymers in addition to one or more hydrophilic polymers. These can be liquids or solids at room temperature. This secondary polymer can be hydrophobic or hydrophilic, although preferably it is hydrophobic. Examples of hydrophilic materials include, but are not limited to, polysaccharides, polyethers, polyurethanes, polyacrylates, cellulosics, and alginates. Examples of hydrophobic materials include, but are not limited to, polyisobutylene, polyethylene-propylene rubber, polyethylene-propylene diene-modified (EPDM) rubber, polyisoprene, styrene-isoprene-styrene, styrene-butadiene-styrene, styrene-ethylene-propylene-styrene, and styrene-ethylene-butylene-styrene. Particularly preferred hydrophobic materials include styrene-isoprene-styrene and styrene-ethylene-butylene-styrene, and even more preferred materials include styrene-isoprene-styrene. The secondary polymer can be in the form of a continuous matrix (i.e., phase) or a discontinuous matrix (e.g., in the form of particles). It can form a bi-continuous or co-continuous phase with the primary hydrophilic polymer. The secondary organic polymer can be elastomeric, thermoplastic, or both. Elastomeric polymers useful as optional secondary polymers in the invention are typically materials that form one phase at 21 degrees Celsius, have a glass transition temperature less than 0 degrees Celsius, and exhibit elastomeric properties. The elastomeric polymers include, but are not limited to polyisoprenes, styrene-diene block copolymers, natural rubber, polyurethanes, polyether-block-amides, poly-alpha-olefins, (C₁-C₂₀) acrylic esters of (meth)acrylic acid, ethylene-octene copolymers, and combinations thereof. The polymer compositions of the present invention can include a wide variety of optional additives. Examples include, but are not limited to, secondary bioactive agents, secondary absorbent particles, foaming agents, swelling agents, fillers, pigments, dyes, plasticizers (for example, mineral oil and petrolatum), tackifiers, crosslinking agents, stabilizers, compatibilizers, extruding aids, chain transfer agents, and combinations thereof.

### A Coating and a Method for Making the Same

Still yet another embodiment of the present invention is a coating comprising the disinfecting agent and a method for making the same. The coating can comprise a film comprising the disinfecting agent. The film of the disinfecting agent may or may not comprise a binder. The coating may or may not be continuous. Moreover, the disinfecting may or may not be one or both of continuously and uniformly distributed throughout the coating. The binder can be any coating binder material. Suitable binders include any organic material, inorganic material, or polymeric material, such as the polymeric materials described herein and/or may include an inorganic binder. The coating may further optionally include additives, such as dispersing agents, fillers, rheology modifiers, leveling agents, spreading agents, adhesion promoters, solvents (including co-solvents), and combinations thereof.

Non-limiting examples of disinfecting coatings include in addition to those indicated above coatings for hospitals and medical facilities, for veterinary facilities, restrooms, dormitories, schools, food processing facilities, embalming facilities, restaurants, residential buildings, agricultural buildings, and public facilities.

In one embodiment, the disinfecting agent particles are blended into any coating system as filler. In another embodiment, the disinfecting agent particles are contacted with the coating after the coating has been applied to a substrate but before the coating has substantially completely dried. The disinfecting agent particles are contacted with the coating a sprinkling or spraying process. Yet other embodiments include the disinfecting agent coatings described herein above.

### An Inorganic Material and a Method for the Same

Another embodiment of the present invention is an inorganic material comprising the disinfecting agent. An inorganic material refers to a metallic alloy, a ceramic or a mineral comprising the disinfecting agent. The disinfecting agent may be alloyed with any one or more non-rare earth metal to form a rare earth-containing alloy. The disinfecting agent may be alloyed with one or more non-rare earth metals by any method known with the metallurgical arts. The disinfecting agent can retain at least some, if not most or all, of its chemical and/or physical properties within the alloy to chemically and/or physically deactivate infectious matter.

Furthermore, the disinfecting agent may be incorporated within and/or form a coating on ceramic material, such as, an inorganic crystalline oxide material, inorganic non-crystalline oxide material or a combination thereof formed form one or more of quartz, feldspar, kaolin clay, china clay, clay, alumina, silica, mullite, silicate, kaolinite, ball clay, bone ash, steatite, petuntse, alabaster, zirconia, carbide, boride, silicide, and combinations thereof. The disinfecting agent may be incorporated within and/or coated onto a ceramic by any method known within the art of material science.

In another embodiment, the disinfecting agent may be chemically and/or physically supported on any mineral, such as, but not limited to quartz, feldspar, kaolin clay, china clay, clay, alumina, silica, mullite, silicate, kaolinite, ball clay, bone ash, steatite, petuntse, alabaster, zirconia, carbide, boride, silicide, talc, and combinations thereof. The disinfecting agent may be chemically and/or physically adhered to and/or combined with any mineral by method known with the chemical and/or mineralogical art.

Standard conditions can mean the solvent is water, including any aqueous based stream and/or source. In other instances, standard conditions can mean conducted and/or extrapolated standard thermodynamic conditions. In yet other instances, standard conditions can mean under process optional conditions, such as, under one or more of temperature, pressure, ionic strength, fugacity, free energy,

As used herein medical includes veterinary, dental, and (human) medical applications, including without limitation preventive, interventional, trauma, non-trauma, home health care, public health (practice and programs), equipment, facilities, expendable and non-expendable equipment, pharmaceuticals, implants and devices, and ancillary products used within the practice of the medial arts.

The present invention provides for methods of using the foregoing rare earth-containing compositions to prevent disease and/or infection to an epithelial tissue (e.g. a mucosal tissue or the skin) comprising applying an effective amount of the composition to the surface or coating an article which is intended to come into contact with the skin or a mucosal tissue. Examples of against which protection may be afforded include, but are not limited to, those induced by biological disease and/or infection causing agents. Specific examples of products that may comprise one or more rare earth-containing compositions to prevent disease and/or infection may include, but are not limited to, means for hair removal (e.g. depilatories, waxing and razors), hair relaxants (e.g. sodium hydroxide, calcium hydroxide, thioglycolates), antiperspirants (e.g. aluminum chlorhydrate and other aluminium salts), dermatological treatments (e.g. alpha hydroxy acids (AHAs), especially glycolic and trichloroacetic acids), keratoyltic skin-irritating conditions (e.g. psoriasis, dandruff, etc.), infectious skin irritants (e.g. bacteria and fungi), and agents applied for therapeutic purposes. The epithelial surface to be protected from irritation may be dermal or mucosal, including vaginal, anorectal, oral or nasal.

The invention further provides for methods of protecting against infection comprising applying, to an epithelial tissue such as the skin or a mucous membrane of the body, an effective amount of one of the foregoing rare earth-containing compositions. Examples of infectious agents against which protection may be afforded include, but are not limited to, infectious agents associated with sexually transmitted diseases, including Human Immunodeficiency Virus (HIV), Human Papilloma Virus (HPV), Herpes Simplex Virus (HSV), Chlamydia trachomatis, Neisseria gonorrhoea, Trichomonas vaginalis, and Candida albicans, as well as infectious agents that may be encountered in a health care setting, including but not limited to Staphylococcus aureus, Pseudomonas aeruginosa, Streptococcus pneumoniae, Escherichia coli, Salmonella typhimurium, Enterococcus, and Neisseria meningitidis, HIV, varicella virus and Hepatitis viruses (e.g., A, B, and C).

In certain alternative non-limiting embodiments of the present invention, the formulations and/or coatings of the invention lack an antimicrobial agent selected from the group consisting of iodophors, iodine, benzoic acid, dihydroacetic acid, propionic acid, sorbic acid, methyl paraben, ethyl paraben, propyl paraben, butyl paraben, cetrimide, quaternary ammonium compounds, including but not limited to benzalkonium chloride, dequalinium chloride, biguanides such as chlorhexidine (including free base and salts (see below)), chloroeresol, chlorxylenol, benzyl alcohol, bronopol, chlorbutanol, ethanol, phenoxyethanol, phenylethyl alcohol, 2,4-dichlorobenzyl alcohol, thiomersal, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, and pharmaceutically acceptable salts thereof.

### Examples

The following examples are provided to illustrate certain embodiments of the invention and are not to be construed as limitations on the invention.

### Example I

Each test in this study contacted about one gram of ceria (CeO₂) powder with 20 mL of 10⁹ pfu/L adenovirus type 2, the contact time was about 24 hours. The virus population (10⁹ pfu/L) is 100 times the NSF test population of 10⁷ pfu/L.

Enumeration of viable virus from de-ionized water samples indicated the ceria powder efficiently decreased the adenovirus by at least about 5 log₁₀. Further analysis by qPCR indicated that adsorption of the adenovirus ranged from about 2.9 to 4.2 log₁₀ removal. The qPCR analysis suggested that adsorption of the adenovirus by the ceria powder was responsible for nearly all the decrease in the virus population. Moreover, it is possible that the qPCR also detected adenovirus genetic material that was adsorbed to ceria fines that was not removed from solution during centrifugation, resulting in the small difference between enumeration and qPCR analyses. One the other hand, it is possible that non-adsorbed genetic material from damaged (that is, non-viable) virus was detected during the qPCR analysis of the supernatant solution.

The loading of adenovirus on the ceria powder was about 2×10⁷ pfu per gram of ceria. The loading value for the adenovirus is about 25% of the loading value observed for MS2/fr. The MS2/fr study had a breakthrough in a column at about 4 log₁₀ removal. The loaded media was extracted with either beef broth or ammonium phosphate. Viable virus was found during extraction. The viable virus level was at or below virus detection levels (that is, much less than about 1%). However, qPCR analysis did detect adenovirus genetic material: 3-5% in phosphate (with a possibly anomalous 51% result) and 1-2% in beef broth extract. The loading and qRCR results did not significantly differ. Furthermore, the recovery of 51% of the virus in the phosphate extraction was not judged to be completely out of the ordinary due to the challenges of working in biological systems.

TABLE 1
Log₁₀ Adsorption of Adenovirus 2 in DI Water to
Ceria Powder and Recovery Following Extraction

| | Ammonium phosphate | | | | Beef Extract | | | |
|---|---|---|---|---|---|---|---|---|
| Trial (#) | 1 | 2 | 7 | Mean | 8 | 9 | 10 | Mean |
| Log₁₀ Adsorption | 5.3 | >5.3 | 5.5 | >5.3 | 5.1 | >5.5 | >5.5 | 5.4 |
| (TCID50) | | | | | | | | |
| Log₁₀ Adsorption (qPCR) | 3.1 | 2.9 | 3.9 | 3.3 | 3.7 | 3.8 | 4.2 | 3.9 |
| Recovery of Adsorbed | 0 | und.* | 0 | 0 | 0 | 0 | 0 | 0 |
| Virus (TCID50) | | | | | | | | |
| Recovery of Adsorbed | 51 | 5 | 3 | 20 | 1 | 2 | 1 | 1.3 |
| Virus (qPCR) | | | | | | | | |

*No detectable virus in supernatant or eluant

In similar studies the ceria powder was shown to have anti-algae, anti-bacteria, and anti-viral properties as follows:

TABLE 2
Log₁₀ Reductions in Algae, Bacteria and
Viruses to Ceria Powder

| | | |
|---|---|---|
| Microbe | Algae (Chlorella) | >4 log |
| | | removal |
| | Bacteria (Klebsiella | >6 log |
| | terrigena) | removal |
| | Bacteria (MRSA) | >6 log |
| | | removal |
| | Virus | >4 log |
| | (MS2/fr/adenovirus) | removal |

### Example II

From about 0.2 to about 0.5 g of paper pulp fibers and about 2 g of cerium oxide powder having an average particle size from about 30 to about 50 microns were mixed together in a 15 mL plastic centrifuge tube. About 7 mL of water was added to the mixture and the tube was shaken vigorously from about 10 to about 30 seconds. The tip of the plastic centrifuge was cut off to form an orifice having a diameter of about 2 mm. The paper pulp formed a filter bed. The water drained through the filter bed with little loss of cerium oxide particles to form a filter media comprising paper pulp fibers and ceria oxide.

### Example III

About 100 grams of cerium carbonate (obtained from HEFA) was slurried with about 600 of water to form an aqueous suspension of the cerium carbonate. The aqueous suspension of cerium carbonate was charged to a 1 liter 316 stainless steel autoclave fitted with 2,000 psi burst disc. The autoclave was sealed and heat was applied to the autoclave. Sufficient heat was applied to the autoclave to maintain the autoclave at a temperature of about 200° C. for about 2 hours. The autoclave pressure was the pressure attained by heating about 600 ml of water within the 1 liter autoclave at a temperature of about 200° C. The autoclave was not stirred and/or agitated. After the 2 hour period at 200° C., the autoclave was cooled and a first sample was collected. The particle size distribution of first sample was determined by a Microtrac® analysis and is shown in FIG. 7. The first sample had a MV of about 11.63 μm, a MN of about 0.16 μm, a MA of about 0.33 μm, and an SD of about 1.56.

A portion of the first sample was dried. The dried first sample was calcinated in a muffle furnace at about 300° C. for about 3 hours to form a calcinated first sample. The particle size distribution of calcinated first sample was determined by a Microtrac® analysis and is shown in FIG. 8. The calcinated first sample had a MV of about 223 μm, a MN of about 0.35 μm, a MA of about 4.76 μm, and a SD of about 182.6.

Another portion of the first sample was left quiescent for about 24 hours, after which the particles remaining suspended were collected as an aqueous sample. The particle size distribution of the aqueous sample was determined by a Microtrac® analysis and is shown in FIG. 9. The aqueous sample had a MV of about 0.26 μm, a MN of about 0.22 μm, a MA of about 0.24 μm, and a SD of about 0.07.

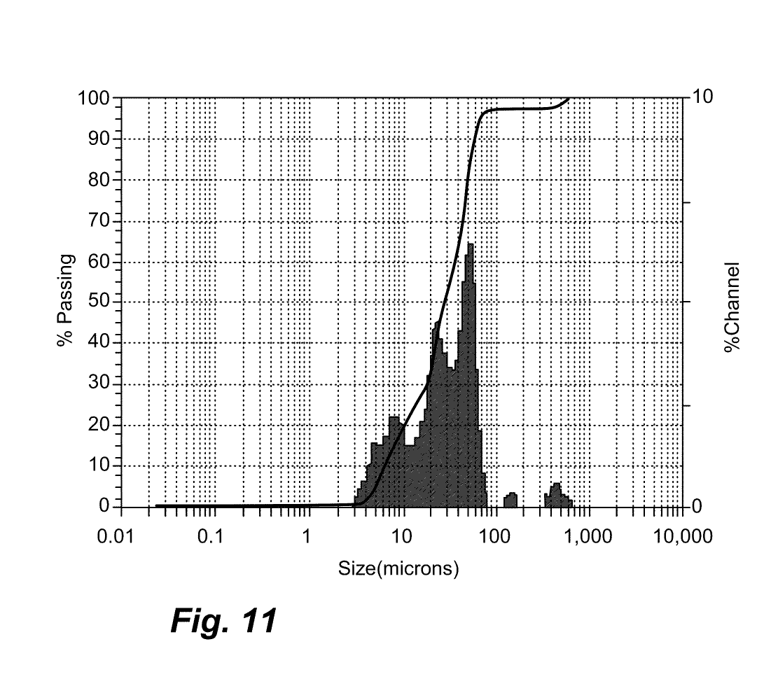

The aqueous sample was calcinated in a muffle furnace at about 300° C. for about 3 hours to form a calcinated second sample. The particle size distribution of calcinated second sample was determined by a Microtrac® analysis and is shown in FIG. 11. The calcinated second sample had a MV of about 21 μm, a MN of about 0.15 μm, a MA of about 0.3 μm, and a SD of about 15.

### Example IV

Example IV is a control process for Example III, wherein about 100 grams of cerium carbonate (obtained from HEFA) was suspended in h about 600 ml of water to form an aqueous suspension of cerium carbonate.

The aqueous suspension of cerium carbonate remained quiescent for about 3 hours, after which a first control sample was collected. The particle size distribution of first control sample was determined by a Microtrac® analysis and is shown in FIG. 11. The first control sample had a MV of about 44.94 μm, a MN of about 6.35 μm, a MA of about 18.08 μm and a SD of about 23.51.

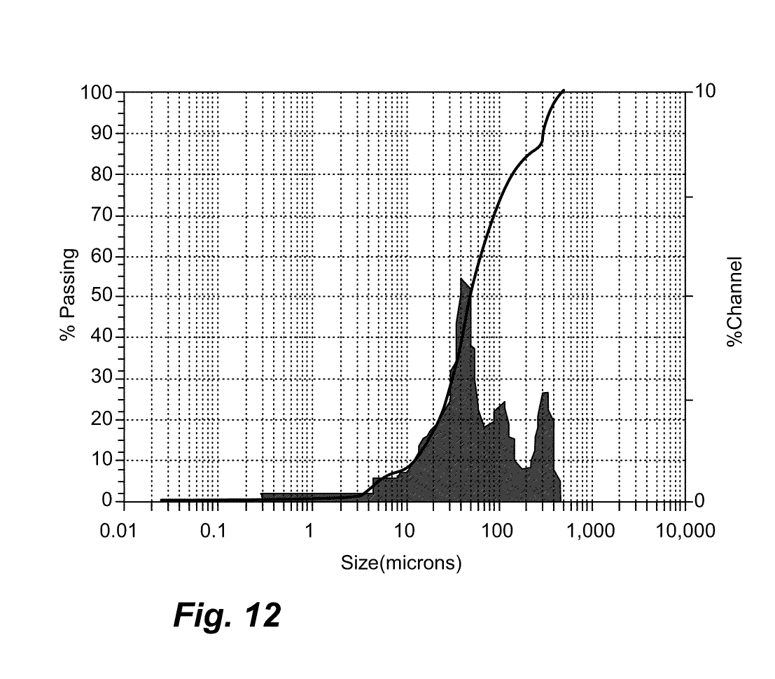

A portion of the first control sample was dried. The dried first control sample was calcinated in a muffle furnace at about 300° C. for about 3 hours to form a calcinated first control sample. The particle size distribution of the calcinated first control sample was determined by a Microtrac® analysis and is shown in FIG. 12. The calcinated first control sample had a MV of about 94.33 μm, a MN of about 0.35 μm, a MA of about 19.96 μm, and a SD of about 84.04.

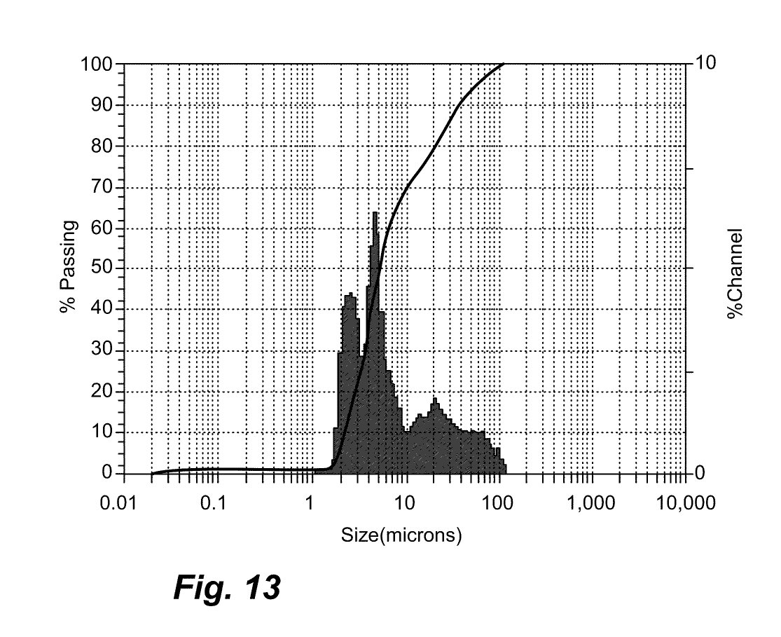

Another portion of the first control sample left quiescent for about 24 hours, after which the particles remaining suspended were collected as a control aqueous sample. The particle size distribution of the control aqueous sample was determined by a Microtrac® analysis and is shown in FIG. 13. The control aqueous sample had a MV of about 13.97 μm, a MN of about 2.78 μm, a MA of about 4.98 μm, and a SD of about 11.45.

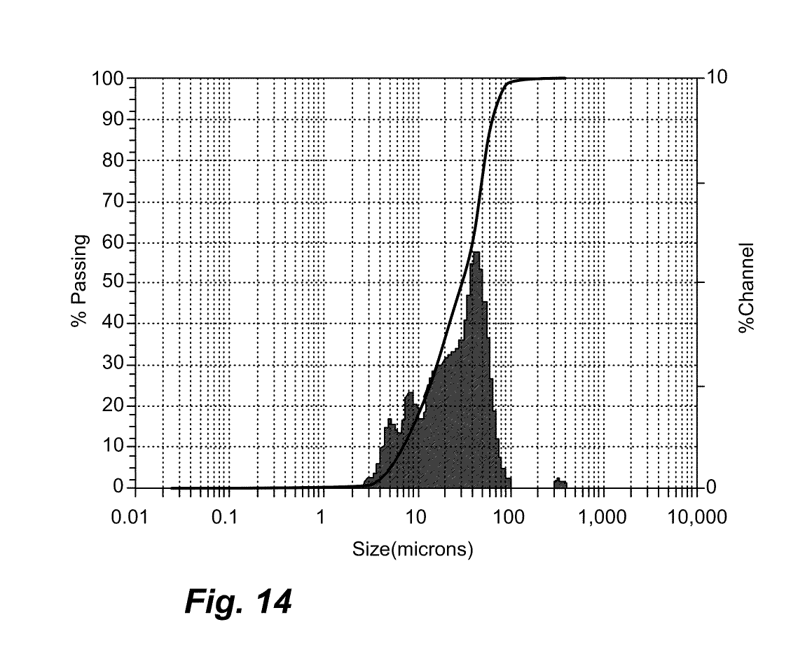

A portion of the first control sample was sonicated for about 2 hours to form a sonicated sample. The particle size distribution of the sonicated sample was determined by a Microtrac® analysis and is shown in FIG. 14. The sample had a MV of about 33.38 μm, a MN of about 6.02 μm, a MA of about 17.21 μm, and a SD of about 21.48.

### Example V

Paper pulp and cotton fibers templates were soaked in de-ionized water to “swell” the fibers. After “swelling” the fibers, the fibers are soaked in a 40 wt % cerium nitrate, Ce(NO₃)₃, to absorb the cerium nitrate into the fibers. Cerium nitrate-containing fibers were heated in a tube furnace under the following conditions: a fifteen minute temperature ramp from about 70 degrees Celsius to about 100 degrees Celsius; about a 50% increase in temperature every hour to final temperature of about 400 degrees Celsius; maintain at about 400 degrees Celsius for about 30 minutes; and followed by about a 3 hour cool down.

A brittle fibrous material having a surface area of less than about 5 m²/g was obtained. The material was calcined at about 700 degrees Celsius. The calcinated material had a surface area of about 5 m²/g.

### Example VI

About 1.56 grams of cotton fiber and about 0.61g paper pulp were soaked in de-ionized water for about 45 minutes before being soaked in the cerium nitrate solution for about 17 hours to form cerium nitrate-containing fibers. The cerium nitrate-containing fibers were heated in a tube furnace at 160 degrees Celsius. The material substantially maintained a fibrous template. However, it was brittle.

A number of variations and modifications of the invention can be used. It would be possible to provide for some features of the invention without providing others.

The present invention, in various embodiments, configurations, or aspects, includes components, methods, processes, systems and/or apparatus substantially as depicted and described herein, including various embodiments, configurations, aspects, sub-combinations, and subsets thereof. Those of skill in the art will understand how to make and use the present invention after understanding the present disclosure. The present invention, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease and\or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments, configurations, or aspects for the purpose of streamlining the disclosure. The features of the embodiments, configurations, or aspects of the invention may be combined in alternate embodiments, configurations, or aspects other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the invention.

Moreover, though the description of the invention has included description of one or more embodiments, configurations, or aspects and certain variations and modifications, other variations, combinations, and modifications are within the scope of the invention, e.g., as may be within the skill and knowledge of those in the art, after understanding the present disclosure. It is intended to obtain rights which include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method, comprising:
contacting one or more rare earth-containing compositions with an infectious biological matter having a first infectious biological matter population, wherein the contacting of the one or more rare earth-containing compositions with the infectious biological matter forms a second infectious biological matter population, the second infectious biological matter population being less the first infectious biological matter population, wherein at least one of the one or more rare earth-containing compositions comprises particles or particulates having an average particle or particulate size from about 50 nanometers to about 1,000 microns and an average surface area of at least about 1 m²g⁻¹.

2. The method of claim 1, wherein the infectious biological matter comprises at least one of a bacterium, a protozoa, a virus, a fungi, a prion or a mixture thereof.

3. The method of claim 1, wherein the one or more rare earth-containing compositions further comprise a device, wherein the device comprises at least one of a textile, an item of apparel, a medical device, a therapeutic formulation, a cleaning composition, a cellulosic-containing material, a polymeric material, a coating material, an inorganic material, or a combination thereof.

4. The method of claim 3, wherein the device comprises one of a woven or non-woven textile, wherein the item of apparel is worn by animal, including a human, wherein the cleaning composition comprises a fluid or solid having at least one surfactant, wherein the cellulosic-containing material comprises at least one of a paper, a cotton, wood, a wood-containing product, or combination thereof product, and wherein the polymeric product comprises one of a homo-polymer, co-polymer, block-polymer, polymeric mixture, polymeric alloy, or a combination thereof comprising at least one of a polyacetal, a polyacrylic, a polyanhydride, a polyamide, a polycarbonate, a polydiene, a polyester, a polyhalo-olefin, a polyimide, a polyimine, a polyketone, a polyolefin, a polyoxide, a polyphylene, a polyphosphazene, a polysilane, a polysiloxane, a polystyrene, a polysulfide, a polysulfoamide, a polysulfonate, a polysulfone, a polysulfoxide, a polythianhydride, a polythioamide, a polythiocarbonate, a polythioester, a polythioketone, a polythioimide, a polythiourea, a polythiourethane, a polyurea, a polyurethane, a polyvinyl, cellulose, chitin, keratin, and a combination or mixture thereof.

5. The method of claim 3, wherein the medical device comprises one of a suture, gauze, sponge, swab, dressing, drape, bandage, or a combination thereof.

6. The method of claim 3, wherein the medical device comprises one of a stapler, surgical instrument, a light-handle cover, medical tubing, medical mesh, an implant, drain component, wound vac component or combination thereof.

7. The method of claim 3, wherein the therapeutic formulation comprises one an aerosol spray, a powder, cream, ointment, slave, liniment, gel, medical solution, wound irrigation system, or combination thereof.

8. The method of claim 1, wherein the infectious biological matter is positioned on or adjacent to an organism, wherein the organism is one of an animal or a plant.

9. The method of claim 8, wherein the animal is one of a human, a domesticated animal, a wild animal, an animal raised as a source of food or income, a companion animal, or a combination thereof.

10. The method of claim 9, wherein the plant is one of a cultivate plant, an uncultivated or wild plant, a plant cultivated for nutritional purposes, plants cultivated for non-food purposes, and combinations thereof.

11. The method of claim 1, wherein the contacting of the one or more rare earth-containing compositions with the infectious matter further comprises:
killing and/or deactivating the infectious biological matter.

12. The method of claim 11, wherein the killing and/or deactivating further comprises:
an interaction of the infectious matter with the one or more rare earth-containing compositions, wherein the interaction comprises one of a chemical interaction, a physical interaction or a combination of a chemical and a physical interaction.

13. The method of claim 13, wherein the second infectious biological matter population divided by the first infectious biological matter population forms a quotient and wherein the quotient is less than about 1.

14. The method of claim 13, wherein the quotient is at most no more than about 10⁻¹, at most no more than about 10⁻², at most no more than about 10⁻³, at most no more than about 10⁻⁴, at most no more than about 10⁻⁵, at most no more than about 10⁻⁶, at most no more than about 10⁻⁷, at most no more than about 10⁻⁸, at most no more than about 10⁻⁹, or at most no more than about 10⁻¹⁰.

15. The method of claim 1, wherein the average surface area is more than about 120 m²g⁻¹.

16. The method of claim 1, wherein the average particle size is from about 0.1 microns to about 10 microns.

17. The method of claim 1, wherein the average particle size is from about 1 micron to about 100 microns.

18. The method of claim 1, wherein one of the one or more rare earth-containing compositions comprises cerium.

19. The method of claim 1, wherein one of the one or more rare earth-containing compositions comprises cerium oxide.

20. The method of claim 1, wherein the one or more rare earth-containing composition comprises at least one of cerium (IV) oxide (CeO₂) and cerium (III) oxide (Ce₂O₃).

21. A method, comprising:
positioning one or more rare earth-containing compositions in a target zone, wherein the target zone has a first population of an infectious biological matter;
contacting the one or more rare earth-containing compositions with the infectious biological matter contained with the target zone; and
killing and/or deactivating the infectious biological matter to form a second population of the infectious biological matter, wherein the second population of the infectious biological matter is less than the first population of the infectious biological matter.

22. The method of claim 21, wherein the infectious biological matter comprises at least one of a bacterium, a protozoa, a virus, a fungi, a prion or a mixture thereof.

23. The method of claim 21, wherein the second infectious biological matter population divided by the first infectious biological matter population forms a quotient of less than about 1.

24. The method of claim 23, wherein the quotient is at most no more than about 10⁻¹, at most no more than about 10⁻², at most no more than about 10⁻³, at most no more than about 10⁻⁴, at most no more than about 10⁻⁵, at most no more than about 10⁻⁶, at most no more than about 10⁻⁷, at most no more than about 10⁻⁸, at most no more than about 10⁻⁹, or at most no more than about 10⁻¹⁰.

25. The method of claim 21, wherein one or more of the rare earth-containing compositions comprise a soluble rare earth-containing composition.

26. The method of claim 25, wherein the water soluble composition has a total dissolved rare earth concentration of at least about 1 M, of at least about 1×10⁻¹ M, of at least about 5×10⁻² M, of at least about 1×10⁻² M, or at least about of at least about 1×10⁻³ M.

27. The method of claim 21, wherein one or more of the rare earth-containing compositions comprise a water insoluble rare earth-containing composition.

28. The method of claim 27, wherein the water insoluble rare earth-containing composition has a total dissolved rare earth concentration of less than about 5×10⁻² M, of less than about 1×10⁻² M, of less than about 1×10⁻³ M, of less than about 1×10^{'14} M, of less than about 1×10⁻⁵ M, of less than about 1×10⁻⁶ M, of less than about 1×10⁻⁷ M, of less than about 1×10⁻⁸ M, of less than about 1×10⁻⁹ M, or of less than about 1×10⁻¹⁰ M.

29. The method of claim 21, wherein the target zone is on or about one of an animal or plant.

30. The method of claim 29, wherein the target zone is one of a wound, an infected wound, a surgical area, an area prone to infection, an area to be protected from the infectious biological matter, an area infected and/or diseased with the infectious biological matter, or a combination thereof.

31. The method of claim 29, wherein the animal comprises a human.

32. The method of claim 29, wherein the animal comprises a domesticated animal, a wild animal, an animal raised as a source of food or income, a companion animal, or a combination thereof.

33. The method of claim 29, wherein the plant is one of a cultivate plant, an uncultivated or wild plant, a plant cultivated for nutritional purposes, plants cultivated for non-food purposes, and combinations thereof.

34. The method of claim 21, wherein the more or more rare earth-containing compositions comprise particles.

35. The method of claim 34, wherein the particles have an average particle size from about 0.1 nanometers to about 1,000 microns.

36. The method of claim 34, wherein the particles have an average surface area of at least about 1 m²g⁻¹.

37. The method of claim 34, wherein the particles have an average surface area of at least about 120 m²g⁻¹.

38. The method of claim 21, wherein cerium comprises at least one of the one or more rare earth-containing compositions.

39. The method of claim 38, wherein the other of the one or more rare earth-containing compositions comprise at least one rare earth element selected from the group of rare elements consisting of La, Nd, Pr, and Sm.

40. The method of claim 21, wherein one of the one or more rare earth-containing compositions comprises cerium oxide.

41. The method of claim 21, wherein the one or more rare earth-containing composition comprises at least one of cerium (IV) oxide (CeO₂) and cerium (III) oxide (Ce₂O₃).

42. An article, comprising:
one or more rare earth-containing compositions; and
one of:
i) a woven textile;
ii) a non-woven textile;
iii) an item of apparel;
iv) a medical device comprising a textile;
v) a medical device comprising a polymer;
vi) a medical device having a polymeric component;
vii) a medical implant;
viii) a therapeutic formulation;
ix) a cleaning composition;
x) a cellulosic-containing material;
xi) a polymeric material;
x) a coating material; and
xi) an inorganic material.

43. The article of claim 42, wherein one or more of the rare earth-containing compositions comprises a soluble rare earth-containing composition.

44. The method of claim 43, wherein the water soluble composition has a total dissolved rare earth concentration of at least about 1 M, of at least about 1×10⁻¹ M, of at least about 5×10⁻² M, of at least about 1×10⁻² M, or at least about of at least about 1×10⁻³ M.

45. The method of claim 42, wherein one or more of the rare earth-containing compositions comprises a soluble rare earth-containing composition.

46. The method of claim 45, wherein the water insoluble composition has a total dissolved rare earth concentration of less than about 5×10⁻² M, of less than about 1×10⁻² M, of less than about 1×10⁻³ M, of less than about 1×10⁻⁴ M, of less than about 1×10⁻⁵ M, of less than about 1×10⁻⁶ M, of less than about 1×10⁻⁷ M, of less than about 1×10⁻⁸ M, of less than about 1×10⁻⁹ M, or of less than about 1×10⁻¹⁰ M.

47. The method of claim 42, wherein the more or more rare earth-containing compositions comprises particles.

48. The method of claim 47, wherein the particles have an average particle size from about 0.1 nanometers to about 1,000 microns.

49. The method of claim 47, wherein the particles have an average surface area of at least about 1 m²g⁻¹.

50. The method of claim 47, wherein the particles have an average surface area of at least about 120 m²g⁻¹.

51. The method of claim 42, wherein cerium comprises at least one of the one or more rare earth-containing compositions.

52. The method of claim 51, wherein the other of the one or more rare earth-containing compositions comprise at least one rare earth element selected from the group of rare elements consisting of La, Nd, Pr, and Sm.

53. The method of claim 42, wherein one of the one or more rare earth-containing compositions comprises cerium oxide.

54. The method of claim 42, wherein the one or more rare earth-containing composition comprises at least one of cerium (IV) oxide (CeO₂) and cerium (III) oxide (Ce₂O₃).

55. A method, comprising:
forming a suspension of a rare earth salt;
charging the suspension to an autoclave;
applying one or both of heat and superatmospheric pressure to the suspension to form an autoclaved suspension;
separating the autoclaved suspension into a liquid phase and a solid phase, and;
calcining one or both of the liquid and solid phases to form rare earth-containing particles.

56. The method of claim 55, wherein the suspension comprises an aqueous suspension.

57. The method of claim 55, wherein the rare earth salt is a substantially insoluble rare earth salt.

58. The method of claim 55, further comprising:
sealing the autoclave prior to the applying of one or both of applying heat and pressure to the suspension.

59. The method of claim 55, wherein the suspension is substantially quiescent during the applying of the one or both of heat and pressure to the suspension.

60. The method of claim 55, wherein one or both of liquid and solid phases are dried prior to calcining.

61. The method of claim 55, wherein the rare earth-containing particles have an average particle size from about 0.1 microns to about 300 microns.

62. The method of claim 61, wherein about 80% of the particles have an average particle size from about 0.1 microns to about 2 microns.

63. The method of claim 55, wherein the rare earth-containing particles have an average particle size from about 0.2 microns to about 0.7 microns.

64. The method of claim 63, wherein about 90% of the particles have an average particle size from about 0.2 microns to about 0.4 microns.
